(19) United States
(12) Patent Application Publication
(10) Pub. No.: US 2021/0106708 A1
(43) Pub. Date: Apr. 15, 2021

(54) THERMORESPONSIVE COMPOSITIONS AND METHODS FOR PREVENTING AND DISRUPTING BIOFILMS

(71) Applicants: CASE WESTERN RESERVE UNIVERSITY, Cleveland, OH (US); THE CLEVELAND CLINIC FOUNDATION, Cleveland, OH (US)

(72) Inventors: Anna Cristina S. SAMIA, Cleveland, OH (US); Salvatore J. FRANGIAMORE, Cleveland, OH (US); Carlos A. HIGUERA RUEDA, Cleveland, OH (US); Alison K. KLIKA, Cleveland, OH (US); Wael K. BARSOUM, Cleveland, OH (US)

(21) Appl. No.: 16/982,090

(22) PCT Filed: Mar. 21, 2019
(86) PCT No.: PCT/US2019/023353

§ 371 Date: Sep. 18, 2020

Related U.S. Application Data
(60) Provisional application No. 62/645,859, filed on Mar. 21, 2018.

(51) Int. Cl. A61L 2/23; A61K 41/00; A61L 27/52; A61L 27/54; A61L 27/44; A61L 2/04

(52) U.S. Cl. CPC A61L 2/23; A61K 41/0052; A61L 27/52; B82Y 5/00; A61L 27/446; A61L 2/04; A61L 27/54

(57) ABSTRACT

One aspect of the present disclosure can include a thermoresponsive nanocomposite for disrupting or preventing biofilm formation. The nanocomposite can include at least one polymer, one or more D-amino acids, and one or more energy-actuatable particles. The nanocomposite can have a first viscosity at about room temperature and, when exposed to about physiological temperature, obtains a second viscosity that is greater than the first viscosity. Application of energy to the nanocomposite from an energy source can excite the one or more energy-actuatable particles to cause localized heat release from the nanocomposite.

### RELATED APPLICATIONS

This application claims the benefit of U.S. Provisional Patent Application Ser. No. 62/645,859, filed Mar. 21, 2018, the entirety of which is hereby incorporated by reference for all purposes.

### GOVERNMENT FUNDING

This invention was made with government support under DMR-1253358 awarded by the National Science Foundation the Federal agency. The government has certain rights in the invention.

### TECHNICAL FIELD

The present disclosure relates generally to anti-biofilm compositions and methods and, more specifically, to nanocomposites and methods for combined thermal and D-amino-acid-assisted biofilm prevention and disruption.

### BACKGROUND

Prosthetic joint infection (PJI) following total joint arthroplasty is a devastating complication requiring surgical intervention and prolonged antimicrobial treatment. In the United States, PJI is reported to be the most common indication for revision total knee arthroplasty (TKA) and the third most frequent indication for revision total hip arthroplasty (THA). Moreover, its prevalence is on the rise, with the projected number of infected THA or TKA procedures in the United States by 2020 exceeding 60,000 and the projected annual cost exceeding $1.62 billion. The underlying pathogenesis of PJI involves the formation of a bacterial biofilm that protects the pathogen from both the host immune response and antibiotics, making it difficult to eradicate such infections. Biofilms on the implants are also resistant to mechanical methods of removal involving brushing/scrubbing, which results in high failure rates of less morbid procedures like irrigation and debridement. Since the removal of biofilm is currently achieved by the explantation of the whole prosthesis, any therapy aimed at destroying the biofilms with retention of the implants may revolutionize the management of PJ.

### SUMMARY

One aspect of the present disclosure can include a thermoresponsive nanocomposite for disrupting or preventing biofilm formation. The nanocomposite can include at least one polymer, one or more D-amino acids, and one or more energy-actuatable particles. The nanocomposite can have a first viscosity at about room temperature and, when exposed to about physiological temperature, obtains a second viscosity that is greater than the first viscosity. Application of energy to the nanocomposite from an energy source can excite the one or more energy-actuatable particles to cause localized heat release from the nanocomposite.

Another aspect of the present disclosure can include a medical implant that is resistant to biofilm formation one or more surfaces thereof. The medical implant can be at least partially coated or impregnated with a thermoresponsive nanocomposite. The nanocomposite can include at least one polymer, one or more D-amino acids, and one or more energy-actuatable particles. The nanocomposite can have a first viscosity at about room temperature and, when exposed to about physiological temperature, obtain a second viscosity that is greater than the first viscosity. Application of energy to the nanocomposite from an energy source can excite the one or more energy-actuatable particles to cause localized heat release from the nanocomposite.

Another aspect of the present disclosure can include a method for disrupting or preventing biofilm formation on a surface. The method can comprise applying energy from an external energy source to a surface that is at least partially coated or impregnated with a nanocomposite. The nanocomposite can include at least one polymer, one or more D-amino acids, and one or more energy-actuatable particles. The nanocomposite can have a first viscosity at about room temperature and, when exposed to about physiological temperature, obtain a second viscosity that is greater than the first viscosity. Energy can be applied to the nanocomposite for a time and in an amount sufficient to excite one or more energy-actuatable particles of the nanocomposite and cause localized heat release from the nanocomposite.

Another aspect of the present disclosure can include a method for disrupting or preventing biofilm formation on an in situ medical implant. One step of the method can include exposing a surface of the implanted medical implant. The surface can be contacted, at about room temperature, with a nanocomposite having a first viscosity. The nanocomposite can include at least one polymer, one or more D-amino acids, and one or more energy-actuatable particles. A period of time can be allowed to pass so that the nanocomposite obtains a second viscosity that is greater than the first viscosity. Energy can be applied to the nanocomposite for a time and in an amount sufficient to excite one or more energy-actuatable particles of the nanocomposite and cause localized heat release from the nanocomposite. The surface of the implanted medical implant can then be covered.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a process flow diagram illustrating a method for disrupting or preventing biofilm formation on a surface according to one aspect of the present disclosure;

FIGS. 2A-F show (A) TEM images of spherical magnetic nanoparticles (s-MNPs) and (B) cubic magnetic nanoparticles (c-MNPs). The corresponding PXRD patterns of the (C) s-MNP and (D) c-MNP samples with reference to the magnetite, Fe₃O₄, iron oxide crystallographic phase. Hydrodynamic sizes of the (E) s-MNP and (F) c-MNP samples measured using DLS, respectively;

FIGS. 3A-B show (A) specific absorption rate (SAR) values evaluated for the s-MNPs and c-MNPs, respectively, measured at different concentrations of Fe (250-750 μg Fe/mL) and variable AC magnetic field amplitudes (1-5 kA/m) excitation at a fixed frequency of 380 kHz. (B) Field-dependent magnetization of the s-MNP and c-MNP samples measured from −1 to 1 T at 300 K using a vibrating sample magnetometer (VSM);

FIG. 4 shows time-dependent HeLa cell cytotoxicity assay results for the s-MNPs and c-MNPs samples in the concentration range from 250 to 750 μg Fe/mL at 2 and 24 h cell exposure times using saline (0.9% NaCl) as positive control;

FIGS. 5A-C show (A) synthetic route for the preparation of the thermoresponsive glycol chitin-based hydrogel from the selective N-acetylation of glycol chitosan. (B) ¹H NMR spectra representing the successful conversion of glycol chitosan to glycol chitin as indicated by the absence of the amine (—NH₂) peak in the spectrum for glycol chitin. (C) FT-IR spectra showing the conversion of the precursor glycol chitosan to glycol chitin as revealed by the loss of the amine peak (—NH₂) and the emergence of the more pronounced carbonyl (—C═O) and amide (—CONH₂) peaks in glycol chitin derivative;

FIGS. 6A-D show (A) rheological measurements of different glycol chitin mixtures in 0.9% NaCl at varying glycol chitin loadings (1-5% wt.) showing the concentration dependence of the viscosity over a temperature range of 5-75° C. (B) Photograph of the synthesized magnetic D-amino acid loaded hydrogel (MagDAA gel containing 5% wt. glycol chitin in 0.9% NaCl) showing its sol-gel transition at the physiological temperature of 37° C. (C) Schematic diagram of the magnetic hyperthermia measurement setup used in the study, wherein the sample to be tested is placed in an insulator in the middle of water cooled AC magnetic field coil, and the temperature is measured using a temperature sensitive fiber optic probe. (D) Magnetic hyperthermia heating profiles of the synthesized c-MNPs dispersed in 0.9% NaCl and suspended in the MagDAA gel matrix (5% wt. glycol chitin solution and 750 μg Fe/mL of c-MNPs) measured at an excitation AC field amplitude (H) of 5 kA/m and a frequency (t) of 380 kHz;

FIGS. 7A-D show (A) concentration dependent biofilm disruption effects of vancomycin, and the corresponding (B) crystal violet stained samples of the remaining biofilms after treatment with different amounts of vancomycin for 24 h. (C) Concentration dependent biofilm disruption effects of D-amino acid mixtures (D-trp, D-tyr, and D-phe) and the corresponding (D) crystal violet stained samples of the remaining biofilm after treatment with different amounts of the D-amino acid mixtures for 24 h. The biofilms were grown from bacterial cultures of S. aureus (2×10⁵ cells/well) in modified tryptic soy broth (3% NaCl, 0.5% glucose) for 24 h at 37° C. An asterisk (*) indicates that the difference in means of the treatments are statistically significant at p<0.05, while NS indicates that the difference in means of the treatments are not statistically significant at p>0.05;

FIGS. 8A-B show TEM images of the synthesized (A) spherical magnetic nanoparticles (s-MNPs) and (B) cubic magnetic nanoparticles (c-MNPs) showing wider viewing area;

FIG. 9 shows S. aureus biofilm disruption activities of vancomycin (Van), bacitracin (Bac), NeutroPhase, and Hibiclens (Hib) after a 24 h incubation period;

FIG. 10 shows Time-dependent in vitro cell cytotoxicity assay of vancomycin (Van), bacitracin (Bac), and NeutroPhase at 2 h and 24 h cell incubation time points. The viability of HeLa cells was determined by exposure to media supplemented by the antibiotics for the specified time (2 h and 24 h) at 37° C. in 5% CO₂. Cell viability was determined using the PrestoBlue assay by measuring absorbance at 570 and 600 nm. The percent viability is represented relative to non-treated controls;

FIGS. 11A-B show (A) the effects of individual D-amino acids against S. aureus biofilm disruption. The biofilms were grown from S. aureus bacterial cell cultures that were incubated overnight and diluted to an optical density (OD) at 595 nm of 0.1 and were further diluted 100×(˜10⁵ CFU/mL) in modified tryptic soy broth (3% NaCl, 0.5% glucose). Each well was filled with 2 mL of the diluted bacterial solution and incubated at 37° C. for an additional 24 h to form complete biofilm coverage. The biofilm dispersal activities of the individual D-amino acids were evaluated and compared relative to the biomass of the positive controls (saline only treatment) by measuring the absorbance of solubilized crystal violet stain at 595 nm following 24 h incubation. An asterisk (*) indicates that the difference of means are statistically significant at p<0.05 while NS indicates that difference of means are not statistically significant at p>0.05. (B) The corresponding crystal violet staining of the remaining biofilms after treatment with the individual D-amino acids; D-methionine (D-met, 50 mM), D-tryptophan (D-trp, 54 mM), D-tyrosine (D-tyr, 2.5 mM), and D-phenylalanine (D-phe, 143.5 mM);

FIGS. 12A-B show (A) time-dependent biofilm disruption effects of the D-amino acid mixture of D-tyr, D-trp, and D-phe against biofilms of S. aureus showing that a 2 h incubation period results in almost 85% biofilm eradication. Dispersive activity of the 200 mM D-amino acid mixture was evaluated using a 1:22:57 molar ratio of D-tyr, D-trp, and D-phe, respectively. (B) The corresponding crystal violet staining of the remaining biofilms after treatment;

FIG. 13 shows Cryo-SEM images of treated (2 h MagDAA gel or 2 h MagDAA gel+10 min AMF) and un-treated (+control) bacterial biofilms. The scale bars of the insets represent 100 μm. The S. aureus biofilms were pre-grown on 400-mesh Formvar-coated Cu grids and imaged using a desktop Phenom ProX SEM operated at 5 kV using a temperature controlled sample holder set at −25° C.;

FIGS. 14A-B show (A) the cumulative release of the D-amino acids in the hydrogel without magnetic field, and (B) with magnetic field actuation, monitored at the OD of 280 nm where the D-amino acids strongly absorb;

FIGS. 15A-B show (A) biofilm disruption activities of lower concentrations of D-AAs with and without AMF excitation, and (B) the corresponding representative crystal violet staining of the remaining biofilms following treatments. The asterisk (*) indicates that the difference of means are statistically significant at p<0.05;

FIG. 16 shows time-dependent HeLa cell cytotoxicity assay results for the c-MNPs, hydrogel (5% wt. glycol chitin in 0.9% NaCl), D-amino acid mixture (D-AA with D-tyr:D-trp:D-phe in 1:22:57 molar ratio), and MagDAA gel at 2 and 24 h cell exposure time periods; NS indicates that the difference in means of the treatments are not statistically significant at p>0.05;

FIG. 17 shows biofilm disruption activity comparison of the different treatments used in this study and the corresponding crystal violet stained samples of the remaining biofilms after the different treatments. The use of the MagDAA gel, which involves initial treatment for 2 h followed by additional AC magnetic field (AMF) treatment for 10 min at AC field amplitude of 5 kA/m and a frequency of 380 kHz, showed complete biofilm disruption. NS indicates that the difference in means of the treatments are not statistically significant at p>0.05;

FIGS. 18A-B show (A) microbial cell viability assay involving luminescence measurement based on the ATP quantification in viable bacterial cells following the addition of the BacTiter-Glo reagent on the cell culture medium after different biofilm treatment conditions; the positive control represents the same number of planktonic S. aureus cells (10⁵) that was used in the formation of the biofilms, and the negative control is the cell culture medium in the absence of any bacterial cells. (B) Colony-forming assay involving the regrow of any remaining adherent bacterial cells following biofilm treatments; the positive control is the untreated biofilm and no biofilm was grown for the negative control. NS indicates that difference of means are not statistically significant at p>0.05;

FIG. 19 shows the effect of magnetic field amplitude of 1 kA/m with different implant materials over 10 minutes of exposure time;

FIG. 20 shows the effect of magnetic field amplitude of 2.5 kA/m with different implant materials over 10 minutes of exposure time;

FIG. 21 shows the effect of magnetic field amplitude of 5 kA/m with different implant materials over 10 minutes of exposure time;

FIG. 22 shows temperature increase and ΔT of cubic shaped iron oxide nanoparticles during magnetic hyperthermia;

FIG. 23 shows the experimental set up of the approach used to study the effect of laser power on different implant materials;

FIG. 24 shows the heating profiles of 0.9% saline with 0.5 W/cm² laser power for 5 and 10 minutes of exposure time;

FIG. 25 shows the heating profiles of 0.9% saline with 0.8 W/cm² laser power for 5 and 10 minutes of exposure time;

FIG. 26 shows the heating profiles of 0.9% saline with 2 W/cm² laser power for 5 and 10 minutes of exposure time;

FIG. 27 shows the heating profiles of metal implants with 0.5 W/cm² laser power over 10 minutes of exposure time;

FIG. 28 shows the effect of laser power of 0.8 W/cm² over 10 minutes of exposure time in comparison to the conditions used in magnetic hyperthermia (right panel);

FIGS. 29A-C show gold nanorods (Au NRs) sample with longitudinal surface plasmon resonance at 810 nm. (A) UV-vis spectra, (B) transmission electron microscopy image and aspect ratio determination, and (C) picture of the sample;

FIG. 30 shows gold nanorods (Au NRs) heating profiles at low laser power (0.8 W/cm²). The AuNRs reached the same ΔT compared to magnetic nanoparticles (green curve);

FIG. 31 shows Crystal violet stain of 1 day-old biofilm (S. aureus) before and after treatments;

FIG. 32 shows colony counting after each treatment to confirm regrow from swab;

FIG. 33 shows AuNRs with different plasmon resonance. The yellow shaded area of the spectra represents the biological “water window”, region where aqueous tissue absorbs relatively little light (700 nm to 1200 nm);

FIGS. 34A-D show Au nanorods that absorb on the excitation wavelength of the NIR AC powered laser (808 nm, represented as a yellow line). (A) UV-VIS spectra of different Au NRs (CTAB capped Au NRs samples). TEM images, scale bar: 200 nm (B) AR: 3.5 Au NRs with longitudinal surface plasmon resonance at 750 nm. (C) AR: 3.7 Au NRs with longitudinal surface plasmon resonance at 810 nm and (D) AR: 4.4 Au NRs with longitudinal surface plasmon resonance at 925 nm;

FIG. 35 shows a comparison of heating and cooling profiles of AuNRs in 0.9% saline solutions with 0.8 W/cm² laser power over 15 minutes exposure time;

FIG. 36 shows surface functionalization of AuNRs following a ligand exchange process;

FIG. 37 shows a study of heating of AuNRs after the ligand exchange in solution and gel phase with an FLIR thermal camera (highlighted in blue) and a temperature probe (highlighted in yellow) at different laser exposure times (laser power is 0.8 W/cm² and Au concentration is 200 ppm);

FIG. 38 shows a study of heating of AuNRs over continuous laser exposure for 15 minutes (laser power is 0.8 W/cm² and Au concentration is 200 ppm);

FIG. 39 shows the effect of Au concentration on heating. More heat can be achieved by increasing the AuNRs concentration (333 ppm) with biocompatible PEG ligands;

FIG. 40 shows cell culture plate dimensions for in vitro study;

FIG. 41 shows the implant and container dimensions used for in vitro study. The treatment containing AuNRs, D-AA loaded in the gel is deposited on the implant;

FIG. 42 shows the scanning microscopic images showing the removal of S. aureus bacterial biofilm attached to the Ti implant surface at different gold concentrations; and

FIG. 43 shows colony re-grow was confirmed from direct swab after each treatments to kanamycin-containing (200 ug/ml) agar gel plate with after 24 hr incubation in oven (37° C.).

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “about” can refer to within an acceptable error range for the particular value as determined by one of ordinary skill in the art, which will depend in part on how the value is measured or determined, i.e., the limitations of the measurement system. Where particular values are described in the application and claims, unless otherwise stated, the term “about” can mean within an acceptable error range for the particular value.

As used herein, the terms “prevent,” “preventing,” and “prevention” with regard to biofilm formation can refer to the inhibition of the development or onset of a biofilm or of a biofilm-related disorder or the prevention of the recurrence, onset, or development of one or more indications or symptoms of a biofilm or of a biofilm-related disorder (e.g., an infection) on a surface or in a subject resulting from the administration of a nanocomposite described herein.

As used herein, the terms “disrupt”, “disrupting”, and “disruption” with regard to a biofilm or biofilm formation can refer to the partial or complete removal of biofilm or biofilm matrix and/or compromise of the integrity of a biofilm including, but not limited to, dispersal of single and mixed bacteria species from the biofilm and/or reduction of bacteria in the biofilm and/or reducing toxic activity associated with the biofilm.

As used herein, the term “eradicate” with regard to a biofilm or biofilm formation can refer to complete removal of biofilm or biofilm matrix. The term “substantially eradicate” can mean removal of at least 50% of biofilm or biofilm matrix, removal of about 50-60% of biofilm or biofilm matrix, removal of about 60-70% of biofilm or biofilm matrix, removal of about 70-80% of biofilm or biofilm matrix, removal of about 80-90% of biofilm or biofilm matrix, or removal of about 90-99% of biofilm or biofilm matrix.

As used herein, the terms “treat”, “treating” or “treatment” can refer to administering a nanocomposite described herein in an amount, manner (e.g., schedule of administration), and/or mode (e.g., route of administration), effective to improve a biofilm-related disorder or a symptom thereof, or to prevent or slow the progression of a biofilm-related disorder or a symptom thereof. This can be evidenced by, e.g., an improvement in a parameter associated with a biofilm or with a biofilm-related disorder or an indication or symptom thereof, e.g., to a statistically significant degree or to a degree detectable to one skilled in the art. An effective amount, manner, or mode can vary depending on the surface, application, and/or subject and may be tailored to the surface, application, and/or subject. By preventing or slowing progression of a biofilm or of a biofilm-related disorder or an indication or symptom thereof, a treatment can prevent or slow deterioration resulting from a biofilm or from a biofilm-related disorder or an indication or symptom thereof on an effected surface or in an affected or diagnosed subject.

As used herein, the terms “biofilm-related disorder”, “biofilm-related disease”, and “biofilm-related condition” can be used herein interchangeably. A biofilm-related disorder can include a disturbance or derangement that affects the normal function of the body of a subject. In some instances, a biofilm-related disorder or disease can be characterized by a disease-related growth of bacteria in that a biofilm is established. One example of a biofilm-related disorder can include dermatological wounds, such as recurring external wounds of bedridden patients and chronic diabetic foot ulcers. Another example of a biofilm-related disorder can include a biofilm-related infection associated with an implanted medical implant. Further, biofilm-related disorders can include co-morbid conditions, such as patients with cystic fibrosis that also have (or are suspected of having) a biofilm.

As used herein, the term “polymer” can refer to natural and synthetic, homopolymers and copolymers comprising multiple repeat units and, unless otherwise indicated, that are linear, branched, or dendritic. In some instances, a polymer can be suitable for use in forming a hydrogel. Examples of such polymers can include natural polymers, such as collagen, cellulose, fibrin, polysaccharides, hyaluronic acid, alginate, chitosan, and derivatives thereof, as well as synthetic polymers, such as poly (ethylene glycol) diacrylate, poly(acryl amide), and poly(vinyl alcohol). Other examples of polymers that can be used to form hydrogels are known in the art. In some instances, the term can refer to a thermoresponsive polymer, which are known in the art and described, for example, in PCT Pub. No. WO 2012/123275.

As used herein, the term “medical implant” can refer to any type of medical implant or device that is totally or partly introduced, surgically or medically, into a subject's body or by medical intervention into a natural orifice, temporarily or for a period of time. Non-limiting examples of medical implants can include jaw bone medical implants, repairing and stabilizing screws, pins, frames (e.g., mesh frames), and plates for bone, spinal medical implants, femoral medical implants, neck medical implants, knee medical implants, wrist medical implants, joint medical implants (e.g., an artificial hip joint), maxillofacial medical implants, limb prostheses for conditions resulting from injury and disease, and combinations thereof. A medical implant can be plastic, metal, or a combination thereof.

As used herein, the term “D-amino acid” can refer to an amino acid that is the chiral form of an L-amino acid. D-amino acids can include natural amino acids, such as histidine, alanine, isoleucine, arginine, leucine, asparagine, lysine, aspartic acid, methionine, cysteine, phenylalanine, glutamic acid, threonine, glutamine, tryptophan, valine, ornithine, proline, selenocysteine, serine and tyrosine. Also, D-amino acids can include non-natural amino acids (i.e., compounds that do not occur in nature but that can be incorporated into a polypeptide chain) and/or amino acid analogs as are known in the art.

As used herein, the term “hydrogel” can refer to a three-dimensional, hydrophilic or amphiphilic polymeric network capable of taking up large quantities of water. The networks can be composed of homopolymers or copolymers, and are insoluble due to the presence of covalent chemical or physical (ionic, hydrophobic interactions, entanglements) cross-links. The cross-links can provide the network structure and physical integrity. Hydrogels exhibit a thermodynamic compatibility with water that allows them to swell in aqueous media.

As used herein, the term “physiological temperature” can refer to a subject's body temperature, which can vary by person, age, activity, and time of day. The average normal body temperature is generally accepted as 98.6° F. (37° C.); however, some studies have shown that the “normal” body temperature can have a wide range, from 97° F. (36.1° C.) to 99° F. (37.2° C.).

As used herein, the term “subject” can refer to a vertebrate, such as a mammal (e.g., a human). Mammals include, but are not limited to, humans, dogs, cats, horses, cows, and pigs.

Overview

Most bacteria can form complex, matrix-containing multicellular communities known as biofilms. Biofilm-associated bacteria are protected from environmental insults, such as antibiotics. However, as biofilms age, nutrients become limiting, waste products accumulate, and it is advantageous for the biofilm-associated bacteria to return to a planktonic existence. Thus, biofilms have a finite lifetime, characterized by eventual disassembly.

Biofilms are understood, very generally, to be aggregations of living and dead micro-organisms, especially bacteria, that adhere to living and non-living surfaces, together with their metabolites in the form of extracellular polymeric substances (EPS matrix), e.g., polysaccharides. The activity of antibiofilm substances that normally exhibit a pronounced growth-inhibiting or lethal action with respect to planktonic cells may be greatly reduced with respect to microorganisms that are organized in biofilms, for example, because of inadequate penetration of the active substance into the biological matrix.

Gram-negative bacteria and Gram-positive bacteria, in addition to other unicellular organisms, can produce biofilms. Bacterial biofilms are surface-attached communities of cells that are encased within an extracellular polysaccharide matrix produced by the colonizing cells. Biofilm development occurs by a series of programmed steps, which include initial attachment to a surface, formation of three-dimensional microcolonies, and the subsequent development of a mature biofilm. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins).

The biofilm can allow bacteria to exist in a dormant state for a certain amount of time until suitable growth conditions arise thus offering the microorganism a selective advantage to ensure its survival. However, this selection can pose serious threats to human health in that biofilms have been observed to be involved in about 65% of human bacterial infections.

The present disclosure includes thermoresponsive nanocomposites and methods for preventing or disrupting biofilms and biofilm formation. Using a nanocomposite of the present application, the inventors of the present disclosure demonstrated the total eradication of S. aureus biofilms, which were resistant to conventional antibiotics and were not completely eradicated by separate D-amino acid or magnetic and photo hyperthermia treatments. Based at least in part on this discovery, the inventors have developed a non-toxic, thermoresponsive nanocomposite containing D-amino acids for sustained release of D-amino acids on targeted infection sites (i.e., sites that include, or are suspected of including, a biofilm) as well as localized heat release upon application of energy to the nanocomposite. Advantageously, the present disclosure provides an externally actuated nanocomposite for thermal treatment following initial biofilm disruption with D-amino acids to ensure complete biofilm eradication and prevention of future bacterial colonization.

Nanocomposites

One aspect of the present disclosure can include a non-toxic thermoresponsive nanocomposite for disrupting or preventing biofilm formation on a surface. The nanocomposite can comprise at least one thermoresponsive polymer, one or more D-amino acids, and one or more energy-actuatable particles. The nanocomposite can have a first viscosity at about room temperature and, when exposed to about physiological temperature, obtain a second viscosity that is greater than the first viscosity. Application of energy to the nanocomposite from an energy source can excite the one or more energy-actuatable particles to cause localized heat release from the nanocomposite.

In some instances, the nanocomposite can be formulated ex vivo so that the nanocomposite has a first viscosity at about room temperature (e.g., about 68° F. to about 77° F.). The first viscosity can be such that the nanocomposite can readily flow over, and cover, a surface—but without washing off completely from the surface—so that the nanocomposite remains on the surface after application thereto. In some instances, the first viscosity can include the nanocomposite being in a sol phase state; that is, an incomplete gel phase state. In some instances, the first viscosity can be less than about 100 Pa·s, about 0 Pa·s to about 100 Pa·s, or about 0 Pa·s to about 75 Pa·s (e.g., about 50 Pa·s).

The nanocomposite having a first viscosity can be applied to, contacted, or treated with (e.g., coated onto) a surface. In one example, the surface is a biological surface, such as a skin surface (e.g., a surface of the epidermis, a surface of the dermis, or a surface of the hypodermis), a muscle surface, a fat surface, a bone surface, a cartilage surface, a tendon surface, or a mucosal surface. In another example, the surface defines at least part of an inanimate object, such as a medical implant.

In some instances, the nanocomposite can obtain a second viscosity that is greater than the first viscosity when exposed to a physiological temperature for a sufficient period of time. The period of time will depend, at least in part, on the composition of the nanocomposite and the location (e.g., an in vivo location, such as an epidermal surface) of the surface upon which the nanocomposite is applied. In one example, the period of time can be less than 60 minutes, between 1 and 24 hours (e.g., at least 2 hours or about 2 hours), or one or more days. The second viscosity can be such that the nanocomposite does not readily flow over or across the surface and, rather, remains essentially fixed in position and shape on or about the surface. In some instances, the second viscosity can include the nanocomposite being in a gel phase state. In some instances, the second viscosity can be greater than about 100 Pa·s but less than about 1000 Pa·s, between about 100 Pa·s and about 750 Pa·s, or about 500 Pa·s.

In another aspect, the nanocomposite can comprise at least one polymer (e.g., a thermoresponsive polymer). In some instances, the nanocomposite can include a plurality of thermoresponsive polymers and comprise a polymer matrix. In one example, polymers used to formulate the nanocomposite can include thermoresponsive polymers known for use in preparing hydrogels including, but not limited to, chitin, hyaluronic acid, alginate, chitosan, cellulose, starch, collagen, gelatin, and the like. In one example, the nanocomposite can be a glycol chitin hydrogel. In some instances, the gelation temperature of the nanocomposite can be tuned by changing the polymer concentration and/or type in the nanocomposite. In one example, the percentage concentration of thermoresponsive polymer(s) in the nanocomposite can be about 1% to about 10% wt./vol., e.g., about 3-5% wt./vol. (e.g., about 5% wt./vol.). In another example, the nanocomposite can comprise a percentage concentration of glycol chitin up to about 8% wt/vol. (e.g., about 5% wt./vol.).

In another aspect, the nanocomposite can comprise one or more D-amino acids. D-amino acids are known in the art and can be prepared using known techniques. Exemplary methods include, e.g., those described in U.S. Publ. No. 20090203091. D-amino acids are also commercially available (e.g., from Sigma Chemicals, St. Louis, Mo. Any D-amino acid can be used in the nanocomposite described herein, including, without limitation, D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, or D-tyrosine.

In some instances, a D-amino acid can be used alone or in combination with other D-amino acids at a concentration that is not biologically toxic. The combinations of D-amino acids can be equimolar or, alternatively, the concentration of each D-amino acid in a mixture of different D-amino acids may be different, i.e., non-equimolar amounts. In one example, the total concentration of three or more D-amino acids in the nanocomposite can be about 50 to about 400 mM (e.g., about 200 mM). In another example, the nanocomposite can include a mixture of at least 3 D-amino acids, such as D-tyrosine, D-tryptophan and D-phenylalanine. In this example, the concentration of each of D-tyrosine, D-tryptophan, and D-phenylalanine can be different. For instance, the concentration of D-tyrosine can be about 0.1 mM to about 3 mM (e.g., about 2.5 mM), the concentration of D-tryptophan can be about 30 mM to about 80 mM (e.g., about 54 mM), and the concentration of D-phenylalanine can be about 100 mM to about 200 mM (e.g., about 143.5 mM). Selection of a particular D-amino acid (or D-amino acids) for the nanocomposite can be based, for example, on the known or suspected type of bacteria species comprising a biofilm and the known structural properties of the D-amino acid(s). For example, selection of D-amino acids having aromatic rings can be structurally advantageous to promote interactions with the protective extracellular polymeric matrix of a biofilm.

In another aspect, the nanocomposite can include one or more energy-actuatable particles. Energy-actuatable particles can include any particle capable of generating heat upon absorption of energy (e.g., electromagnetic radiation, light) from an external energy source. In some instances, energy-actuatable particles are water-soluble. In some instances, energy-actuatable particles can include microparticles or nanoparticles. Microparticles can have a diameter of between about 0.15 to about 10 microns. Nanoparticles can have a diameter of between about 1 to about 100 nm. As discussed below, the presence of one or more energy-actuatable particles as part of the nanocomposite advantageously permits external thermal actuation or stimulation following initial biofilm disruption (with the D-amino acid(s)) to ensure complete (or substantially complete) eradiation and destruction of remaining attached bacterial cells and any planktonic bacterial cells released from disrupted biofilms before they can reattach and form biofilm at the same or a different location.

In one example, energy-actuatable particles can include magnetic nanoparticles. Magnetic nanoparticles can be made of one or a combination of materials capable of being magnetized by an external energy source (e.g., a source of electromagnetic waves, an AC magnetic field, RF waves) so that, upon physical excitation, they can transduce the applied external energy into heat. One example of a material used to make magnetic nanoparticles is iron oxide. In this example, the concentration of magnetic nanoparticles in a nanocomposite of the present application can be about 100 μg Fe/mL to about 1000 μg Fe/mL, e.g., about 250 μg Fe/mL to about 750 μg Fe/mL (e.g., about 750 μg Fe/mL). Magnetic nanoparticles can have spherical or cubic shapes. Spherical nanoparticles can have an average diameter of about 10 to about 50 nm (e.g., about 30 nm). Cubic nanoparticles can have an average diameter of about 10 nm to about 50 nm (e.g., about 25 nm).

In one example, a nanocomposite of the present application can include one or more iron oxide nanoparticles having a cubic shape (e.g., with an average diameter of about 25 nm) and being present in the nanocomposite at a concentration of about 750 μg Fe/mL.

In another example, energy-actuatable particles can include any particle capable of generating heat upon absorption of light from an external energy source. For example, energy-actuatable particles can include plasmonic nanoparticles that generate heat upon absorption of light (e.g., from a laser) in the wavelength range of about 400-1200 nm. Such plasmonic nanoparticles can be made, for example, from gold (e.g., gold nanorods, gold nanocages, gold nanoshells) or silver and silver/gold alloys. In some instances, energy-actuatable particles can include gold nanorods having: an average length of about 50 nm to about 100 nm, e.g., about 70-90 nm (e.g., about 78 nm); an average diameter of about 10 nm to about 50 nm, e.g., about 15 nm to about 25 nm (e.g., about 20 nm); and an aspect ratio of about 1.0 to about 10.0, e.g., about 3 to about 5 (e.g., about 4). In some instances, the gold nanorods can be coated with any type of mono-, bi-, or multi-functional thiol-polyethylene glycol (thiol-PEG), e.g., about 2,000 to about 20,000 in molecular weight (MW) (e.g., about 5,000 MW).

In one example, a nanocomposite of the present application can include one or more gold nanorods at a concentration of about 10 ppm to about 5000 ppm, e.g., about 750 ppm to about 1000 ppm (e.g., about 500 ppm).

In some instances, the nanocomposite can include a combination of different types of energy-actuatable particles. For example, the nanocomposite can include both magnetic nanoparticles and plasmonic nanoparticles.

In some instances, the nancomposite can additionally or optionally include one more antibiotic. The antibiotic(s) can be any compound known to one of ordinary skill in the art that can inhibit the growth of, or kill, bacteria. Non-limiting examples of antibiotics can include: lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); glycopeptide antibiotics (such as vancomycin); polypeptide antibiotics (such as bacitracin); macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Pefloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics are commercially available, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Another aspect of the present disclosure can include a medical implant that is at least partially (e.g., partly or entirely) treated (e.g., coated or impregnated) with the nanocomposite of the present disclosure. In some instances, the medical implant can be treated prior to implantation in a subject. In other instances, the medical implant may have been previously implanted in a subject and then surgically removed, whereafter the medical implant is treated with the nanocomposite and then re-implanted in the subject. In one example, a treated medical implant can include a prosthetic joint, such as a prosthetic hip or knee.

Methods

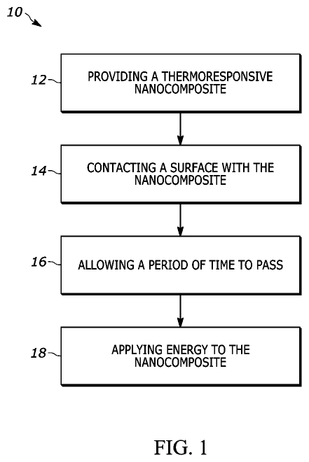

Another aspect of the present disclosure can include a method 10 (FIG. 1) for disrupting or preventing biofilm formation on a surface. The method 10 is illustrated as process flow diagrams with flowchart illustrations. For purposes of simplicity, the method 10 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 10.

The method 10 described herein can be used to prevent or delay the formation of, and/or treat (e.g., eradicate or substantially eradicate), biofilms. In exemplary methods, the biofilms are formed by biofilm-forming bacteria. The bacteria can be a gram-negative bacterial species or a gram-positive bacterial species. Non-limiting examples of such bacteria can include a member of the genus Actinobacillus (such as Actinobacillus actinomycetemcomitans), a member of the genus Acinetobacter (such as Acinetobacter baumannii), a member of the genus Aeromonas, a member of the genus Bordetella (such as Bordetella pertussis, Bordetella bronchiseptica, or Bordetella parapertussis), a member of the genus Brevibacillus, a member of the genus Brucella, a member of the genus Bacteroides (such as Bacteroides fragilis), a member of the genus Burkholderia (such as Burkholderia cepacia or Burkholderia pseudomallei), a member of the genus Borelia (such as Borelia burgdorferi), a member of the genus Bacillus (such as Bacillus anthracis or Bacillus subtilis), a member of the genus Campylobacter (such as Campylobacter jejuni), a member of the genus Capnocytophaga, a member of the genus Cardiobacterium (such as Cardiobacterium hominis), a member of the genus Citrobacter, a member of the genus Clostridium (such as Clostridium tetani or Clostridium difficile), a member of the genus Chlamydia (such as Chlamydia trachomatis, Chlamydia pneumoniae, or Chlamydia psiffaci), a member of the genus Eikenella (such as Eikenella corrodens), a member of the genus Enterobacter, a member of the genus Escherichia (such as Escherichia coli), a member of the genus Francisella (such as Francisella tularensis), a member of the genus Fusobacterium, a member of the genus Flavobacterium, a member of the genus Haemophilus (such as Haemophilus ducreyi or Haemophilus influenzae), a member of the genus Helicobacter (such as Helicobacter pylori), a member of the genus Kingella (such as Kingella kingae), a member of the genus Klebsiella (such as Klebsiella pneumoniae), a member of the genus Legionella (such as Legionella pneumophila), a member of the genus Listeria (such as Listeria monocytogenes), a member of the genus Leptospirae, a member of the genus Moraxella (such as Moraxella catarrhalis), a member of the genus Morganella, a member of the genus Mycoplasma (such as Mycoplasma hominis or Mycoplasma pneumoniae), a member of the genus Mycobacterium (such as Mycobacterium tuberculosis or Mycobacterium leprae), a member of the genus Neisseria (such as Neisseria gonorrhoeae or Neisseria meningitidis), a member of the genus Pasteurella (such as Pasteurella multocida), a member of the genus Proteus (such as Proteus vulgaris or Proteus mirablis), a member of the genus Prevotella, a member of the genus Plesiomonas (such as Plesiomonas shigelloides), a member of the genus Pseudomonas (such as Pseudomonas aeruginosa), a member of the genus Providencia, a member of the genus Rickettsia (such as Rickettsia rickettsii or Rickettsia typhi), a member of the genus Stenotrophomonas (such as Stenotrophomonas maltophila), a member of the genus Staphylococcus (such as Staphylococcus aureus or Staphylococcus epidermidis), a member of the genus Streptococcus (such as Streptococcus viridans, Streptococcus pyogenes (group A), Streptococcus agalactiae (group B), Streptococcus bovis, or Streptococcus pneumoniae), a member of the genus Streptomyces (such as Streptomyces hygroscopicus), a member of the genus Salmonella (such as Salmonella enteriditis, Salmonella typhi, or Salmonella typhimurium), a member of the genus Serratia (such as Serratia marcescens), a member of the genus Shigella, a member of the genus Spirillum (such as Spirillum minus), a member of the genus Treponema (such as Treponema pallidum), a member of the genus Veillonella, a member of the genus Vibrio (such as Vibrio cholerae, Vibrio parahaemolyticus, or Vibrio vulnificus), a member of the genus Yersinia (such as Yersinia enterocolitica, Yersinia pestis, or Yersinia pseudotuberculosis), and a member of the genus Xanthomonas (such as Xanthomonas maltophilia).

Biofilm-producing bacteria, e.g., a species described herein, can be found in a live subject, in vitro, or on a surface (as described herein).

Referring to FIG. 1, one step of the method 10 can include providing a thermoresponsive nanocomposite (Step 12). The nancomposite can be formulated, as described, for a particular application or indication. For example, the nanocomposite can be particularly formulated for application to a specific medical implant and/or where prevention and/or disruption of a particular biofilm (e.g., containing a known species of bacteria) is desired. As described above, the nanocomposite can be initially formulated ex vivo with a first viscosity. The nanocomposite can be formulated, for example, by admixing one or more thermoresponsive polymers, one or more D-amino acids, one or more energy-actuatable particles, and a liquid (e.g., saline).

At Step 14, a surface (e.g., of a medical implant) can be contacted with the nanocomposite having a first viscosity. In some instances, the surface can be contacted with the nanocomposite in an ex vivo setting, e.g., before the medical implant is implanted in a subject. In this case, all or only a portion of the medical implant can treated with the nanocomposite, as discussed above. In other instances, the medical implant can be contacted with the nanocomposite in situ, e.g., where the implant has previously been implanted in a subject. In this instance, the nanocomposite can be contacted with a location of the medical implant that has, or is suspected of having, a biofilm. In further instances, a surface of the medical implant can be contacted with the nanocomposite following removal of the medical implant from a subject.

Upon contacting the nanocomposite with a surface of the medical implant, the physiological temperature of the subject can cause the nanocomposite to obtain a second viscosity that is greater than the first viscosity (Step 16). A period of time is permitted to pass that is sufficient for the nanocomposite to obtain the second viscosity. The period of time can be minutes, hours, or days. In one example, the period of time can be about 1-3 hours, e.g., about 2 hours. During the period of time (and thereafter), the nanocomposite provides sustained release of D-amino acids.

At Step 18, energy from an external energy source can be applied to the nanocomposite in an amount and for a time sufficient to excite one or more energy-actuatable particles and thereby cause localized heat release from the nanocomposite. The type of energy applied to the nanocomposite can depend, for example, on the type of energy-actuatable particles present in the nanocomposite. Where the magnetic nanoparticles are present in the nanocomposite, energy can be applied from an external source of a magnetic field or radio-frequency (RF) wave excitation. In some instances, an applied magnetic field can include a source of alternating current (AC) having an amplitude of about 2 kA/m to about 15 kA/m, e.g., about 1-10 kA/m (e.g., about 5 kA/m) and a frequency of about 200 kHz to about 400 kHz (e.g., about 380 kHz), which is applied for a period of time (such as about 1 min to about 60 minutes, e.g., about 10 minutes) sufficient to excite the magnetic nanoparticles and cause localized heat release from the nanocomposite.

Alternatively, where the energy-actuatable particles comprise plasmonic nanoparticles (e.g., gold nanorods), a laser can be applied to the nanocomposite in an amount and for a time sufficient to excite one or more gold nanorods and cause localized heat release from the nanocomposite. In some instances, an external light source (e.g., a laser) comprising near-infrared radiation (e.g., having a wavelength of about 785 nm to about 1046 nm, e.g., about 810 nm) can be applied. Non-limiting examples of such light sources can include Nd:YAG, laser diode, and Ti:sapphire laser systems. Power applied by the laser can be about 0.1 W/cm² to about 4 W/cm², e.g., about 0.5 W/cm² to about 2 W/cm² (e.g., about 1 W/cm²). The time over which the nanocomposite is exposed to the external energy source (irradiation time) can be seconds or minutes, e.g., about 1 minute to about 30 minutes, e.g., about 5 minutes to about 20 minutes (e.g., about 20 minutes). Alternatively, an external light source having a wavelength in the visible spectrum (e.g., about 420 nm to about 690 nm) can be applied. In one example, a laser having a wavelength of about 808 nm, a spot size of about 1 cm², and a power of about 1 W/cm² can be applied for a time sufficient to excite one or more gold nanorods and cause localized heat release from the nanocomposite. In another example, a laser having a wavelength of about 810 nm and a power of about 1 W/cm² can be applied for about 5-25 minutes (e.g., about 20 minutes) to excite one or more gold nanorods and cause localized heat release from the nanocomposite.

Advantageously, the application of energy to the nanocomposite not only enhances cumulative release of D-amino acids from nanocomposite, but also enables complete disruption of the biofilm (e.g., to eradicate or substantially eradicate planktonic bacterial cells released from the disrupted biofilm and prevent of future bacterial colonization).

In one example, the method 10 can be employed for a subject suffering from (or suspected of suffering from) a prosthetic joint infection (PSJ). PJI, also referred to as periprosthetic infection, is defined as infection involving the joint prosthesis and adjacent tissue. PJI can be treated by a number of different medical and surgical strategies, including open or arthroscopic debridement without removal of the prosthesis, resection of the prosthesis without reimplantation, resection of the prosthesis with reimplantation of a new prosthesis either at the time of removal (one-stage or direct arthroplasty exchange) or delayed by weeks to months (two-stage arthroplasty exchange), arthrodesis, amputation, or antimicrobial suppression without surgery. Even with these treatment approaches, however, a relatively high failure rate persists.

Two-stage arthroplasty exchange includes two surgical stages. Stage 1 includes: (1) re-opening a surgical incision at a site where a medical implant was previously implanted; (2) aggressive debridement of infected tissue with lavage; (3) removing the medical implant, followed by additional debridement and lavage; (4) loading an antibiotic-infused spacer in place of the medical implant; and (5) closing the incision. Stage 2, which can be performed days to weeks later, includes: (1) re-opening the surgical incision; (2) removing the spacer, followed by debridement and lavage; and (3) implanting the revision arthroplasty. A failure rate of about 30% for two-stage arthroplasty exchange persists, however, as repeating the two-step surgical process becomes more challenging due to the fact that remaining bone and tissue is limited and compromised, which can lead to amputation. As discussed below, the present application advantageously provides nanocomposites and associated methods for significantly reducing or eliminating the failure rate currently associated with treating PJI and, in particular, two-step exchange arthroplasty.

In some instances, a nanocomposite of the present application can be administered at the surgical site (i.e., to the infected medical implant, e.g., by injection) prior to Stage 1. Administration can occur, for example, minutes, hours, or days before Stage 1 (e.g., the night before). Then, after the incision is opened (partially or completely) in Stage 1, energy can be applied to the nanocomposite from an external energy source (e.g., a laser) in an amount and for a time sufficient to excite the energy-actuatable particles (e.g., plasmonic nanoparticles) and cause localized heat release from the nanocomposite. The remainder of Stage 1 as well as Stage 2 can then be performed as described above.

In other instances, a two-step exchange arthroplasty can be performed without exchanging the medical implant. In this instance, a nanocomposite that includes one or more antibiotics can be administered to a surface of the medical implant and/or the space surrounding the infected medical implant (e.g., following debridement/lavage in Stage 1). Then, the administered nanocomposite can be removed in Stage 2.

In further instances, a two-step exchange arthroplasty can be performed without exchanging the medical implant. A nanocomposite of the present application can be administered at the surgical site (i.e., to the infected medical implant, e.g., by injection) prior to Stage 1. Administration can occur, for example, minutes, hours, or days before Stage 1 (e.g., the night before). Then, after the incision is re-opened (partially or completely) in Stage 1, energy can be applied to the nanocomposite from an external energy source (e.g., a laser) in an amount and for a time sufficient to excite the energy-actuatable particles (e.g., plasmonic nanoparticles) and cause localized heat release from the nanocomposite. The remainder of Stage 1 as well as Stage 2 can then be performed but without exchanging the medical implant.

The following Examples are for the purpose of illustration only and are not intended to limit the scope of the claims, which are appended hereto.

### Example 1

Experimental Section

Materials and Reagents

The following chemicals were purchased from Sigma-Aldrich and used as received: D-tyrosine (99%), D-tryptophan (≥98.0%), D-phenylalanine (≥98%), D-methionine (≥98%), vancomycin hydrochloride, acetic acid (glacial), methanol (≥99.8%), ethanol (70%), toluene (99.9%), hexane (≥98.5%), acetic anhydride (≥99%), acetone (≥99.5%), glycol chitosan (≥50%), iron(III) chloride hexahy-drate (98%), iron(III) acetylacetonate (97%), oleic acid (90%), 4-biphenylcarboxylic acid (95%), benzyl ether (98%), 1-octadecene (90%), triethylamine (99%), and trimethylamine N-oxide (98%). Sodium hydroxide (99%), ammonium hydroxide (14.8 N), 1-butanol (≥99.4%), crystal violet, Dulbecco's Modified Eagle Medium (DMEM), fetal bovine serum (FBS), penicillin/streptomycin (10 000 U/mL), trypsin-EDTA (0.25%) phenol red, phosphate buffered saline (PBS) solution, sodium chloride solution (0.9%, w/v), LB broth powder (50% tryptone, 25% yeast extract, 25% sodium chloride), LB agar powder (25% tryptone, 12.5% yeast extract, 25% sodium chloride and 37.5% agar) and PrestoBlue assay kit were purchased from Fisher Scientific. Sodium oleate (>97%) was obtained from TCI America. The ligand, 3-(triethoxysilyl)-propyl succinic anhydride was purchased from Gelest, Inc. S. aureus (ATCC 10832) and HeLa cells (ATCC CCL-2) were purchased from the American Type Culture Collection. Hibiclens (Mölnlycke Health Care Inc.), Neutrophase (NovoBay Pharmaceuticals, Inc.), and bacitracin (Sandoz) were obtained from Dr. Higuera-Rueda. BacTiter-Glo microbial cell viability assay was purchased from Promega.

### Synthesis of Spherical Magnetic Nanoparticles (s-MNPs)

s-MNPs were prepared via a two-step synthetic approach: the first step involved the thermal decomposition of an iron oleate complex to form wüstite (FeO) nanoparticles, which was then subsequently converted to the magnetite (Fe₃O₄) phase using a mild oxidation step (Bhattarai, N. et al., Adv. Drug Delivery Rev. 62 (1), 83-99). The iron oleate precursor was prepared by dissolving iron(III) chloride hexahydrate (FeCl₃.6H₂O, 40 mmol) and sodium oleate (120 mmol) in a flask containing deionized (DI) water (60 mL), ethanol (80 mL), and hexane (140 mL). The mixture was then heated at reflux for 4 h. After reflux, the organic layer containing the iron oleate precursor was separated from the aqueous layer and washed three times with warm water to remove salt byproducts and excess reagents. The iron oleate mixture was then dried under vacuum for 72 h and stored for further use.

To synthesize 30 nm s-MNPs, iron oleate (3.6 g) and oleic acid (15 mL) were vigorously stirred under argon (Ar) atmosphere. The solution was then heated to 380° C. (3° C./min) and refluxed for an hour. The reaction mixture was then cooled to ambient temperature and FeO nanoparticles were precipitated via the addition of 1:1 toluene:ethanol solvent mixture (35 mL) and centrifuged at 7000 rpm for 20 min. FeO nanoparticles were converted to Fe₃O₄ using trimethylamine N-oxide [(CH₃)₃NO] as an oxidizing agent. Briefly, (CH₃)₃NO (0.1 mmol) was added to FeO nanoparticles (100 mg), oleic acid (0.5 mL), and 1-octadecene (20 mL). The reaction mixture was heated to 130° C. (10° C./min) for 2 h and the temperature was further raised to 280° C. (10° C./min) and held at that temperature for 1 h. The nanoparticles were then cooled down to ambient temperature and transferred to a 50 mL centrifuge tube. A 30 mL solvent mixture of 1:1 toluene:ethanol was added to the reaction mixture and centrifuged at 7000 rpm for 20 min. The s-MNP precipitate collected was dissolved in 10 mL of toluene, degassed with Ar, and stored for further use.

### Synthesis of Cubic Magnetic Nanoparticles (c-MNPs)

c-MNPs were synthesized using a thermal decomposition approach (Bauer, L. et al., Nanoscale 8 (24), 12162-12169). Iron(III) acetylacetonate (0.5 mmol), 4-biphenyl-carboxylic acid (0.5 mmol), oleic acid (1.90 mmol), and benzyl ether (52.61 mmol) were placed in a 50 mL three-neck round-bottom flask. The mixture was heated for 30 min at 70° C. under an Ar atmosphere, then again for 90 min at 300° C. After the reaction mixture has cooled down to 60° C., ethanol was added and the nanoparticles were centrifuged, isolated, and redispersed in toluene.

Characterization of Magnetic Nanoparticles (MNPs)

The MNP size and shape were evaluated by transmission electron microscopy (TEM). TEM samples were prepared by placing 5 μL of a dilute suspension of the MNPs on a 400 mesh Formvar-coated copper grid and allowing the solvent to evaporate slowly at room temperature. TEM images were obtained with a FEI Tecnai G2 Spirit BioTWIN transmission electron microscope operated at 120 kV. The mean particle size and size distribution were evaluated by measuring at least 200 nanoparticles for each sample. The crystal structure of the samples was analyzed by powder X-ray diffractometry (PXRD) performed in a Rigaku MiniFlex powder X-ray diffractometer using Cu Kα radiation (λ=0.154 nm). For the XRD analysis, the diffraction patterns were collected within a 2θ range of 25-75°. The average hydrodynamic radii of the different oxidized MNP samples were measured by dynamic light scattering (DLS) on a ZetaPALS particle size analyzer (Brookhaven) at a scattering angle of 90°. The total Fe concentration in each sample was measured using a fast sequential atomic absorption spectrophotometer (AAS) Varian 220FS AA. For the elemental Fe analysis, the samples were digested in concentrated hydrochloric acid overnight to completely dissolve the MNPs.

The magnetic hyperthermia measurements were performed using a MSI Automation bench mount magnetic induction heating system. The MNPs samples were exposed to an alternating current (AC) magnetic field excitation with variable magnetic field amplitude (H) of 1-5 kA/m at a fixed frequency (f) of 380 kHz. All samples were measured inside insulated NMR glass tubes with an internal diameter of 7.5 mm, at different iron concentrations (250-750 μg/mL). To evaluate the temperature profiles of the samples upon excitation with a continuous alternating magnetic field, the change in temperature of the samples was monitored with a fiber optic temperature probe (Neoptix T1) and was recorded every 5 s. Prior to turning the magnetic field on, the sample temperature was recorded for 30 s to obtain a stable baseline for the calculation of the SAR values. The SAR was calculated from the initial slope over the first 30 s of the heating curve using the equation:

$$SAR = \frac{{CV}_{s}}{m} \frac{dT}{dt}$$

where dT/dt is the initial slope of the heating curve, C is the volumetric specific heat capacity of the solvent, V_{S} is the volume of the sample, and m is the mass of magnetic material (i.e., g of Fe) in the sample. The reported average SAR values were calculated from three repeat measurements. The field-dependent magnetization data of the MNPs were measured using a Lakeshore 7307 vibrating sample magnetometer (VSM) from −1 to 1 T at 300 K.

### Synthesis of Glycol Chitin Hydrogel

A thermoresponsive hydrogel based on glycol chitosan was prepared according to a previously reported procedure (Li, Z. et al., Carbohydr. Polym. 92(2), 2267-2275). Briefly, glycol chitosan (0.25 g) was dissolved in a 50:50 mixture of water to methanol (50 mL), followed by the slow addition of acetic anhydride (0.83 mL). The resulting reaction mixture was then left to stir for 48 h. The product was then precipitated with acetone and centrifuged at 17000 rpm for 10 min at 4° C. The isolated glycol chitin was then redissolved in deionized water and incubated in 1 M NaOH solution for 12 h to remove undesired reaction byproducts. The sample was then dialyzed in a dialysis membrane (molecular weight cut off=2000 Da) for 3 days. The purified product was then reprecipitated with acetone and centrifuged at 4° C. to isolate the glycol chitin. Following lyophilization, the product was collected and stored for further use. The successful conversion of glycol chitosan to glycol chitin was confirmed via ¹H NMR spectroscopy using a 500 MHz Bruker Ascend Avance III HD. Moreover, FT-IR spectroscopy measurements were performed using a Thermo Scientific Nexus 870 ATR-FTIR spectrometer in the spectral range of 600-4000 cm⁻¹.

Rheological studies on glycol chitin hydrogels prepared by mixing different mass loadings of glycol chitin in 0.9% NaCl were performed by measuring temperature dependent changes in viscosity of the hydrogels using a Thermo Scientific HAAKE MARS III Rheometer. The viscosities of the hydrogels were measured in the temperature range of 5-75° C. at a heating rate of 0.0575° C./s and a shear rate of 0.1/s.

### Preparation of Magnetic Hydrogels Using Water-Soluble c-MNPs

Magnetic hydrogels were prepared by mixing water-soluble c-MNPs with the glycol chitin-based hydrogels at the appropriate loading concentrations. Water-soluble c-MNPs were prepared by modifying the surface of the as-prepared oleic acid coated nanoparticles with a carboxyl-terminated silane ligand using a previously reported ligand exchange process (Situ, S. et al., ACS Appl. Mater. Interfaces 6 (22), 20154-20163; Burke, D. et al., Int. J. Mol. Sci. 16 (10), 23630-23650). A solution containing NH₄OH in 1-butanol (1 M), triethylamine (1.4 mL), deionized water (0.5 mL), and 3-(triethoxysilyl)propyl succinic anhydride (100 μL) was vortexed for 5 min. The c-MNPs in toluene (25 mg/mL) were added to the previous solution and vortexed for another 5 min. The sample was allowed to sit for at least 1 h after which the c-MNPs transferred from the organic to the water phase. The resulting mixture was then centrifuged at 8500 rpm for 30 min. The supernatant was removed and the c-MNP sample was redispersed in deionized water.

Biofilm Formation and Dispersal Assays

S. aureus were cultured overnight in agar plates or in lysogeny broth (LB) with agitation (200 rpm) at 37° C. Biofilm formation was evaluated under static conditions using 12-well plates (Falcon, USA). Bacterial cultures made overnight were diluted to an OD₅₉₅ of 0.1 and further diluted 100× (˜10⁵ CFU/mL) in modified tryptic soy broth (3% NaCl, 0.5% glucose) and each well was filled with 2 mL of the diluted bacterial solution and incubated at 37° C. for 24 h. Biofilm dispersal activities were evaluated by removing the culture media from the biofilms after 24 h and replacing it with saline solution (0.9% NaCl) containing either individual or a combination of amino acids at the specified concentrations (pH 7.4), or the other antimicrobial agents used in the clinic (vancomycin, bacitracin, NeutroPhase, Hibiclens). After exposure to the different treatment methods, the plates were gently washed with phosphate buffered saline (PBS, 1×) thrice and then stained with 500 μL of 0.1% (w/v) crystal violet for 30 min. The wells were then washed with PBS and the crystal violet stain was solubilized with 30% acetic acid. The solution was diluted 20 times and biofilm biomass was estimated by measuring the crystal violet stained biofilm remnants, which absorbs at 595 nm.

Magnetic Hyperthermia-Aided Biofilm Dispersal Assays

For samples treated with magnetic hyperthermia, the biofilms were grown on individual 35×10 mm polystyrene cell culture plates. Magnetic nanoparticles or magnetic glycol chitin-based, D-amino-acid-loaded hydrogel (MagDAA gel) were incorporated in the treatment solutions. Each cell culture plate was placed in the middle of a water-cooled coil and exposed to an alternating current (AC) magnetic field with amplitude (H) of 5 kA/m and a frequency (f) of 380 kHz. All assays were repeated in triplicates.

Cryo-Scanning Electron Microscopy (SEM) Analyses of Biofilms

S. aureus biofilm samples for cryo-SEM analyses were grown on Formvar-coated 400 mesh copper grids, following the procedure previously described. The preformed biofilms were then subjected to the different treatments and fixed using ice-cold methanol for 20 min prior to analysis on a Phenom ProX SEM operated at 5 kV. The treated and untreated biofilms were placed on the SEM sample holder using conductive carbon tape and a temperature controlled sample holder that was set and maintained at −25° C.

Cell Viability Assays

HeLa cells were cultured in Dulbecco's Modified Eagle Medium supplemented with 10% fetal bovine serum and 1% penicillin/streptomycin at 37° C. in 5% CO₂ in a humidified atmosphere. The cells were seeded to 100% confluence in clear 96-well tissue culture plates (Costar, Corning, N.Y., USA) for 24 h. The cells were then exposed to the different treatments in media and incubated for the specified time. After treatment, the cells were washed in sterile phosphate buffered saline and incubated in 100 μL/well of DMEM (10% FBS, 1% penicillin/streptomycin). Cell viability was evaluated using the PrestoBlue assay (Invitrogen, USA), following the manufacturer's instructions. Briefly, 10 μL of PrestoBlue assay reagent was directly added to each well and the plates were incubated at the cell culture incubator (37° C. and 5% CO₂) for 2 h. The absorbance at 570 and 600 nm were then measured using a SpectraMax i3 microplate reader (Molecular Device Inc., Sunnyvale, Calif.). All assays were performed in triplicates.

Luminescent Assay for Microbial Cell Viability

Bacterial cell cultures of S. aureus (2×10⁵ cells/well) in modified tryptic soy broth (3% NaCl, 0.5% glucose) were added to 12-well plates and incubated for 24 h at 37° C. After incubation, the cell culture medium was removed and 2 mL of MagDAA gel was added to the appropriate samples and the sample was further incubated for 2 h (n=6) and then treated with 10 min of AMF-induced hyperthermia. After treatment, the supernatant was collected (including loosely bound cells) from the individual wells and then centrifuged at 13000 rpm for 1 min. The bacterial cells were then dispersed in 1 mL of PBS and 100 μL of the bacterial cell sample was plated in an opaque white 96-well plate. 100 μL of the BacTiter-Glo reagent was added to each well and the sample was mixed thoroughly and incubated for 5 min before luminescence measurements were performed. All measurements used to quantify luciferase activity were performed using a luminescent multiplate reader (MPL2 and Orion Microplate Luminometer, Berthold Detection Systems, Pforzheim, Germany). To eliminate the interference of the luciferase reaction with the solvent, additional negative control containing only the cell culture medium without bacterial cells was included in the measurement.

Colony-Forming Assay to Evaluate the Survival of Remaining Adherent Microbial Cells

The S. aureus bacterial biofilms treated with MagDAA gel for 2 h or subjected to combined MagDAA gel+AMF treatments were individually scraped using sterile cotton swabs, and the swabs were separately incubated in 10 μL deionized water with 990 μL modified LB broth. A 20 μL aliquot of the incubated cell culture medium from each of the incubated swabs were then evenly dispersed into pre-prepared LB agar gel plates, and the LB agar gel plates were then incubated at 37° C. for 24 h to check the regrowth of bacterial colonies following treatments.

Statistical Analyses Statistical analyses were performed using one-way ANOVA with a Tukey test to determine statistical difference between the groups (p<0.05).

Results

### Synthesis and Characterization of Magnetic Nano-Particles (MNPs)

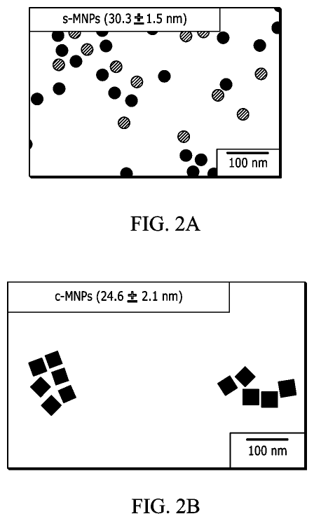

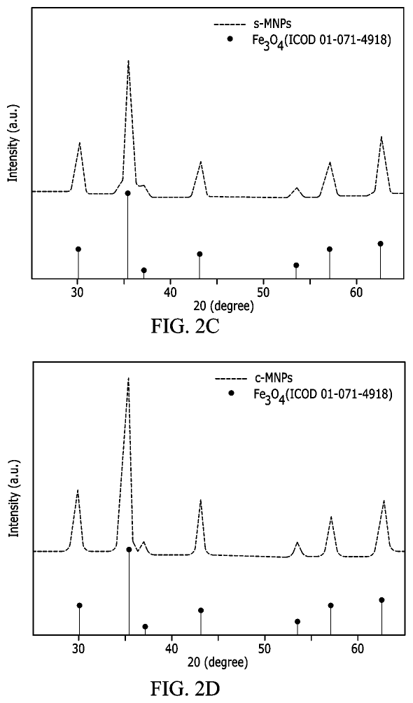

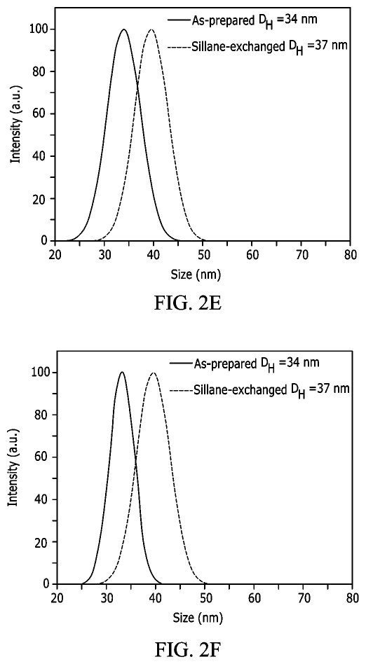

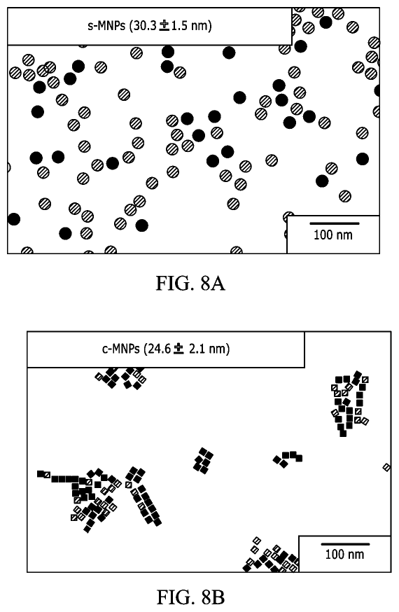

The use of magnetic hyperthermia for heat treatment-aided biofilm disruption involving iron oxide nanoparticles, which release localized heat upon excitation with an AC magnetic field, was achieved by first optimizing the heating efficiency of the magnetic nanoparticles (MNPs). To accomplish this task, magnetic nanoparticles with spherical (s-MNPs) and cubic (c-MNPs) shapes with similar volumes were synthesized by adapting previously reported thermal decomposition synthetic methods with slight modifications (Park, J. et al., 2004 Nat. Mater. 3(12), 891-895; Situ, S. et al., 2014 ACS Appl. Mater. Interfaces 6 (22), 20154-20163; Bauer, L. et al., 2016 Nanoscale 8 (24), 12162-12169). Shown in FIGS. 2A-B are the transmission electron microscope (TEM) images of the synthesized s-MNPs that have an average diameter of 30 nm and the prepared c-MNPs with an average size of 25 nm, respectively; wider view TEM images of the MNPs are presented in FIGS. 8A-B. The powder X-ray diffraction (PXRD) patterns obtained from the samples show that both types of MNPs have the magnetite (Fe₃O₄) crystallographic phase (FIGS. 2C-D). Moreover, results from dynamic light scattering (DLS) measurements showed that the as-prepared oleic acid coated-MNPs did not significantly change in size (based on the evaluated hydrodynamic diameters) after conversion to their water-soluble forms using a silane-based ligand exchange process (Situ, S. et al., 2014 ACS Appl. Mater. Interfaces 6 (22), 20154-20163; Burke, D. et al., 2015 Int. J. Mol. Sci. 16 (10), 23630-23650 (FIGS. 2E-F).

The magnetic hyperthermia performance of the synthesized MNPs was evaluated by measuring the change in temperature under AC magnetic field excitation at different sample concentrations (250-750 μg Fe/mL) and under varying AC field amplitudes (H from 1-5 kA/m) at a constant excitation frequency (f) of 380 kHz. In our study, the values of H and f (H×f≤1.90×10⁹ A m⁻¹ s⁻¹) were judiciously selected to avoid possible side effects, such as damage to non-infected cells, because of excess heat production by MNPs at much higher magnetic fields and excitation frequencies; this value is within the range of the acceptable threshold limit for clinical magnetic hyperthermia that avoids any adverse effects to healthy cells. Moreover, the tolerable magnetic field strengths for thermotherapy range from 3.8 to 13.5 kA/m.

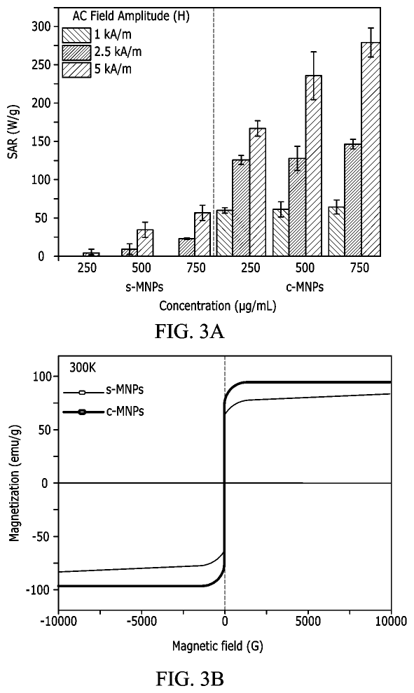

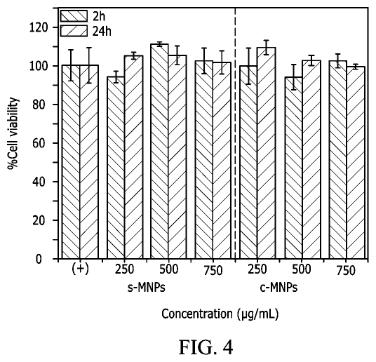

The heating efficiency was assessed by estimating the specific absorption rate (SAR) based on the obtained temperature curves during AC field exposure. Comparison of the magnetic hyperthermia performance of the differently shaped MNPs showed that the cubic sample (c-MNPs) has higher heating efficiency (larger SAR values across the concentration range) than its spherical counterpart (s-MNPs) (FIG. 3A). This result can be both attributed to the enhanced shape anisotropy in the cubic shaped MNPs and its higher saturation magnetization (FIG. 3B) that was evaluated using vibrating sample magnetometry (VSM), which together lead toward enhanced magnetic hyperthermia performance. Since the c-MNPs at a concentration of 750 μg Fe/mL demonstrated the best heating efficiency, this cubic MNP formulation was used in the succeeding preparation of the magnetic hydrogels for biofilm disruption. Moreover, to prepare for the in vitro cell studies, the biocompatibility of the water-soluble MNPs was also assessed through a PrestoBlue cell viability assay using HeLa cells. The results of the cell toxicity assay show that both s-MNPs and c-MNPs samples are nontoxic in the concentration range of 250-750 μg Fe/mL for both 2 and 24 h cell exposure times (FIG. 4), and can thus be used in the following cell experiments.

### Preparation of Thermoresponsive Magnetic Glycol Chitin Hydrogel

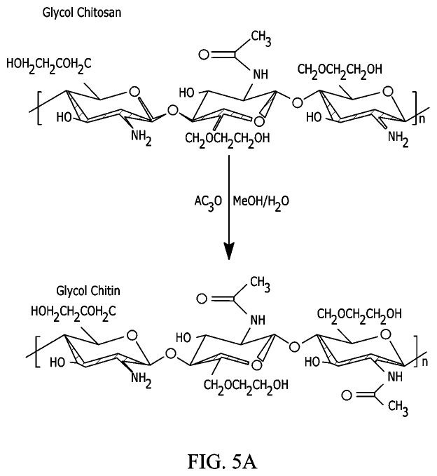

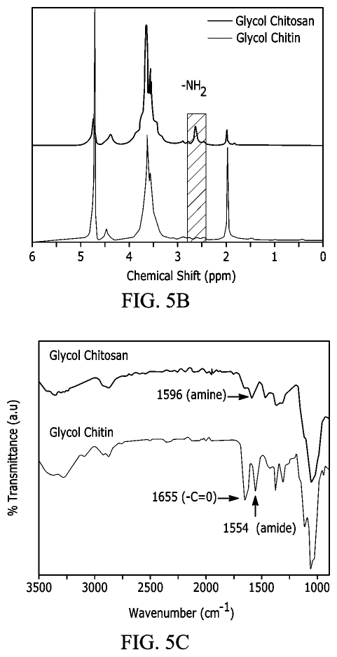

We prepared a thermoresponsive magnetic glycol chitin based hydrogel that is nontoxic and has a gelation temperature that can be tuned by changing the concentration of glycol chitin solution loading. The glycol chitin polymer was prepared via the controlled N-acetylation of the glycol chitosan precursor (FIG. 5A). The successful conversion of the glycol chitosan precursor to the desired glycol chitin derivative can be confirmed from the obtained ¹H NMR spectra from the two molecular forms, which show the loss of the amine group signal following successful conversion to glycol chitin (FIG. 5B). FT-IR spectroscopy also confirms the formation of the glycol chitin derivative, which is reflected by the loss of the amine peak and the appearance of stronger peaks for the carbonyl and amide groups (FIG. 5C).

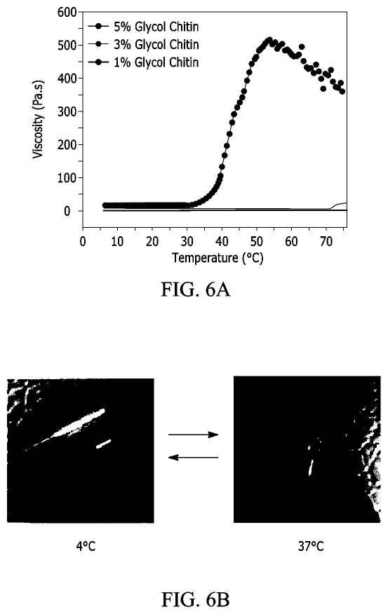

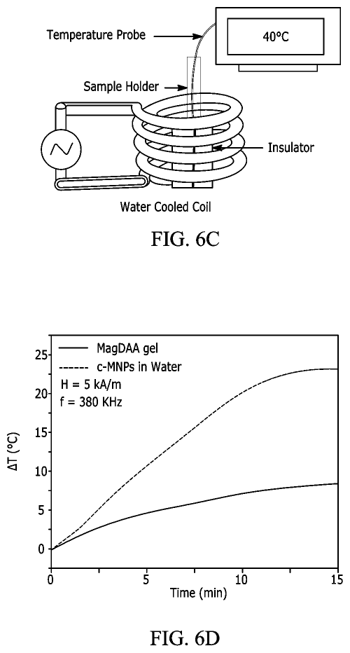

After verifying the successful conversion of glycol chitosan to the glycol chitin derivative, the changes in viscosity as a function of glycol chitin mass loading across a temperature range of 5-75° C. were evaluated using a Thermo Scientific HAAKE MARS III Rheometer. The rheological measurement data obtained for different glycol chitin mixtures in 0.9% NaCl at varying glycol chitin loadings (1-5% wt.) are shown in FIG. 6A. The 5% wt./vol glycol chitin solution showed an abrupt rise in viscosity at around the physiological temperature of 37° C., which signifies the onset of the sol to gel phase transition. In contrast, lower mass loadings (1 and 3% wt./vol. glycol chitin solutions) barely showed changes in viscosity across the temperature range indicating that the gelation process was not achieved at these low glycol chitin polymer concentrations at the specified temperature range (FIG. 6A). The concentration and temperature dependent sol-gel transition at 37° C. was further confirmed through the tube inversion method where sample gelation was characterized by absence of flow for 30 s (FIG. 6B).

Using the 5% wt. glycol chitin solution (with sol-gel transition at 37° C.) and 750 μg Fe/mL c-MNPs (MNP with highest heating efficiency) as sample matrix, a magnetic glycol chitin-based, D-AA loaded hydrogel (MagDAA gel) nano-composite was prepared. The magnetic hyperthermia property of the resulting MagDAA gel nanocomposite was evaluated and compared to the water dispersed c-MNPs (FIGS. 6C-D). It can be observed that a lower increase in temperature was achieved in the hydrogel sample (5° C.) compared to when the MNPs are in solution (25° C.). The decrease in heating efficiency of the c-MNPs in the gel matrix can be attributed to the viscous nature of the hydrogel whereby Brownian rotation is restricted leading to lower heating performance (FIG. 6D).

Biofilm Disruption Using Magnetic Glycol Chitin-Based, D-Amino-Acid-Loaded Hydrogel (MagDAA Gel) Nanocomposite

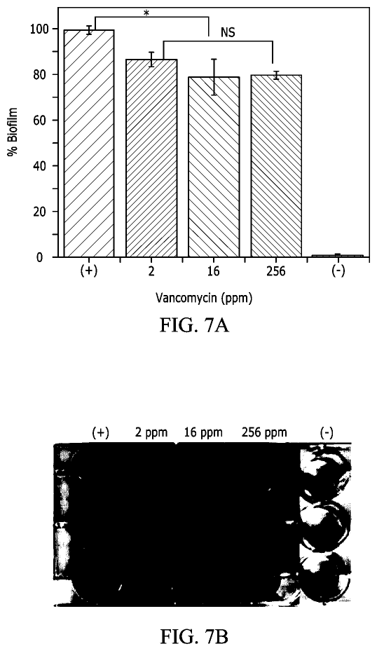

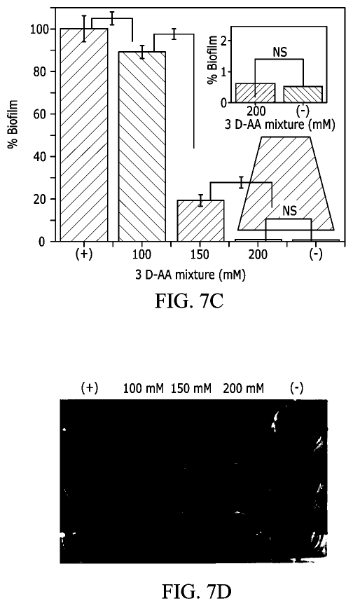

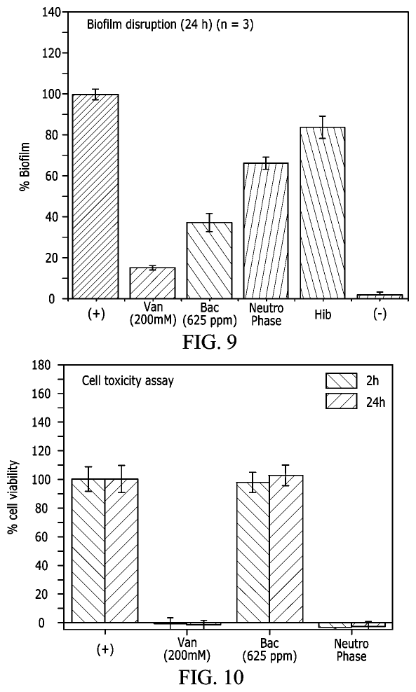

S. aureus is one of the major causes of hospital- and community-based acquired infections resulting in the formation of methicillin-resistant S. aureus (MRSA) strains that is more difficult to treat using antibiotics that are currently available. In our study, one of the most widely used antibiotics in the clinical setting, vancomycin, was evaluated for its biofilm dispersal activity against S. aureus at relevant clinical concentrations (16 mg/kg or ppm every 12 h for vancomycin), as well as concentrations that are lower and higher than the clinical dosage to establish if there is a concentration effect. FIGS. 7A-B show that vancomycin at concentrations ranging from 2 to 256 ppm do not disrupt preformed biofilms. In addition to the above concentrations of vancomycin, we further explored the biofilm disruption property of vancomycin at a much higher concentration (200 mM), as well as the dispersal properties of other clinically used antibacterial reagents, such as bacitracin, NeutroPhase, and Hibiclens, after a 24 h incubation period (FIG. 9). Even though vancomycin (200 mM) showed promising results within 24 h, the in vitro cytotoxicity assay proved that 200 mM of vancomycin is extremely toxic to mammalian cells to be of use in the clinical setting (FIG. 10).

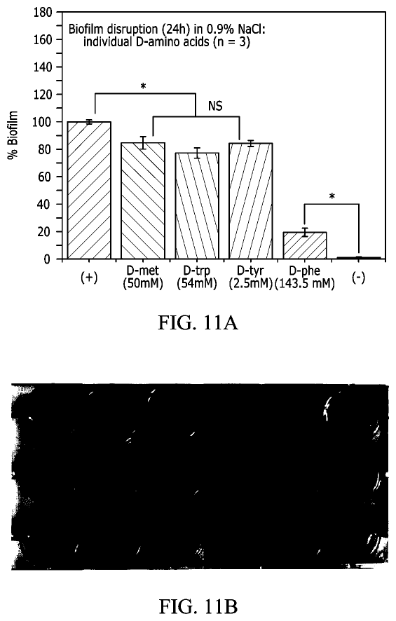

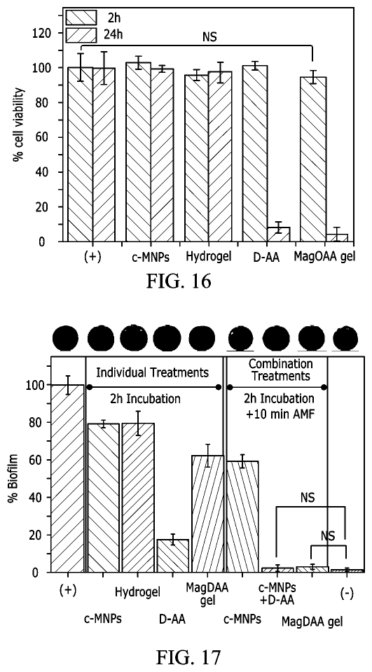

Considering the structural properties of the D-AAs, we hypothesized that having aromatic rings on the D-AAs would be structurally advantageous to promote interactions with the protective extracellular polymeric matrix of the biofilms, and as such, we investigated the effects of individual D-AAs on bacterial biofilm disruption (FIGS. 11A-B). The results showed that none of the individual D-AAs were able to implement the complete eradication of the biofilms at their highest possible water-soluble concentrations. In this regard, we evaluated the biofilm dispersal activity of a mixture of three aromatic D-AAs (D-trp, D-tyr, and D-phe) at concentrations close to the sum of their maximum individual solubilities (100-200 mM) (FIGS. 7C-D). Increasing biofilm disruption activity was observed with increasing amino acid concentration, and total biofilm disruption was observed at a concentration of 200 mM. To test for biocompatibility, HeLa cell toxicity assays were performed on the D-AA and antibiotic treatments. Results showed that the D-AA mixture at a concentration of 200 mM was toxic to HeLa cells at 24 h incubation period (FIG. 16).

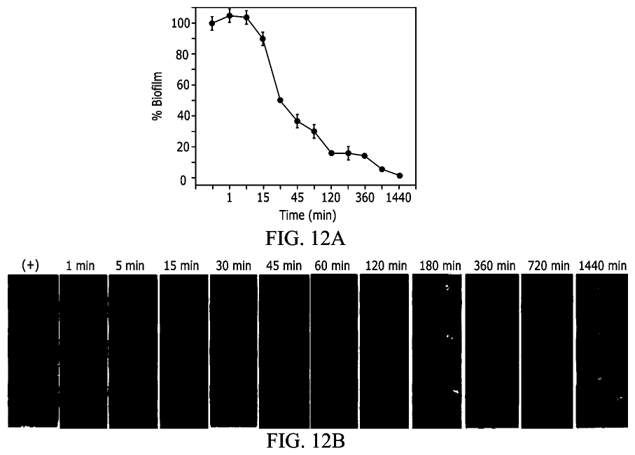

From this result, we decided to perform a time dependent biofilm disruption assay using 200 mM D-AAs (15 min to 24 h) to find a viable incubation time point that shows significant biofilm disruption, as well as nontoxicity to HeLa cells (FIG. 16 and FIGS. 12A-B). FIGS. 12A-B show that biofilm disruption starts to plateau after 2 h with ˜85% disruption activity. In addition, the 2 h incubation of HeLa cells with the 200 mM D-AA mixture demonstrates that it is nontoxic at this incubation time point (FIG. 16). In contrast, at a similar vancomycin concentration of 200 mM, the antibiotic showed extreme cell toxicity for both at 2 and 24 h incubation periods (FIG. 10).

Considering the results of the biofilm disruption and cell toxicity assays, we then formulated our MagDAA gel with 200 mM loading of D-AAs consisting of 54 mM D-typ, 2.5 mM D-tyr, and 143.5 mM D-phe. A combination of 2 h D-AA biofilm disruption and 10 min application of an alternating magnetic field (AMF-induced hyperthermia at H=5 kA/m; f=380 kHz) showed complete biofilm disruption with the appropriate positive (saline treatment) and negative (no bacterial cell treatment and no biofilm formation) controls (FIG. 17). In contrast, individual treatments with D-AA or magnetic hyperthermia have not led to complete biofilm eradication (FIG. 17). The observed incomplete biofilm eradication by magnetic hyperthermia can be attributed to the selected low field (H) and frequency (f) values that were adopted to avoid damage to healthy cells. On the other hand, hydrophobic D-AAs have been known to permeabilize bacterial lipid cell membrane causing cell wall disruption, but not complete biofilm damage. Therefore, in our study, we have adapted an adjuvant treatment approach using a combination of D-AA pretreatment (for initial disruption of the EPS and microbial cell associations) followed by magnetic thermal therapy, to completely destroy remaining bacterial biofilms. In order to investigate the effects of the D-AA and combined D-AA+AMF-induced hyperthermia treatment on the biofilm disruption, we have performed cryo-scanning electron microscopy (cryo-SEM) analyses on treated and untreated preformed biofilms (FIG. 13). Our cryo-SEM studies demonstrate the disruptive effects of the D-AAs on the EPS of the biofilms and the complete eradication of the biofilms upon additional AMF-induced hyperthermia treatment (FIG. 13).

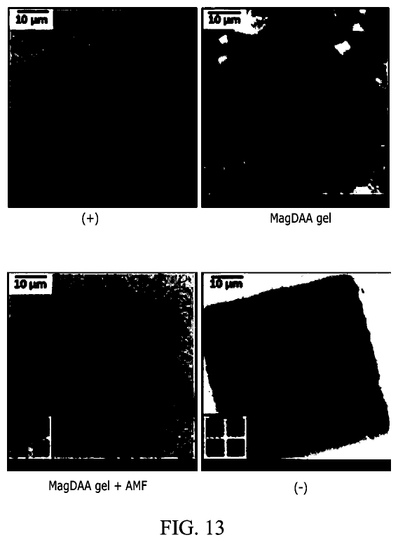

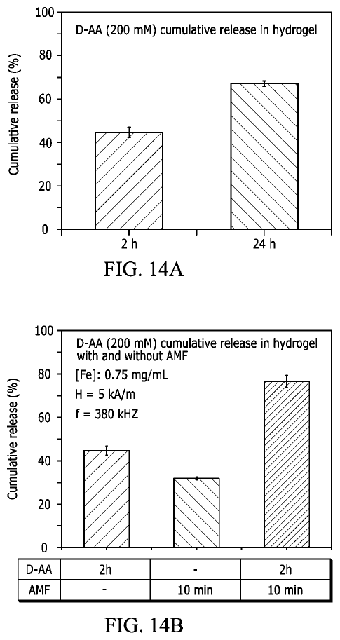

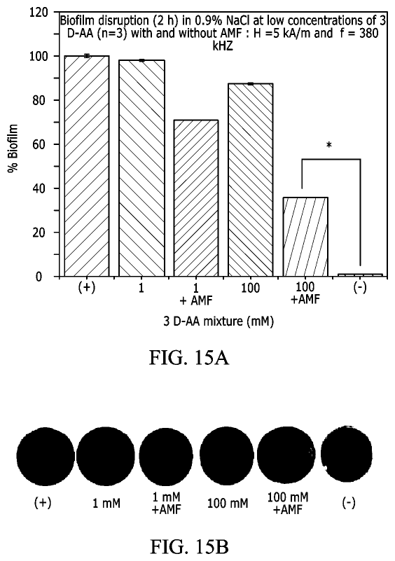

The magnetic hyperthermia effect of the c-MNPs in the MagDAA gel served two purposes; enhancement of the cumulative release of the D-AAs in the gel, which was estimated by monitoring the absorbance of the released D-AA mixture at 280 nm (FIGS. 14A-B), and enabling the complete disruption of the biofilm after pretreatment with the D-AA mixture (FIG. 17). Moreover, cell toxicity assay showed that 2 h exposure of HeLa cells to the MagDAA gel is nontoxic to the cells (FIG. 16). We have also investigated the effect of using lower concentrations of D-AAs and found that only the 200 mM concentration combined with AMF treatment leads to complete biofilm eradication (FIGS. 15A-B). While our in vitro HeLa cell toxicity studies indicate nontoxicity of the 200 mM D-AA mixture at a 2-h incubation period, the actual release of the D-AAs from the gel matrix is only up to a total of ˜70% or 140 mM for both the 24 h incubation time point or the combined 2 h+AMF treatment approach (FIGS. 14A-B). From the recent in vivo studies conducted by Harmata et al. (2015 Clin. Orthop. Relat. Res. 473(12), 3951-3961) using a sheep animal model, they have demonstrated that D-AA mixture concentrations are nontoxic in vivo from 0 to 200 mM for a 24 h incubation period and also do not inhibit new bone formation. Our treatment approach is within the range of the reported in vivo concentration tolerance for D-AA with minimal side effects.

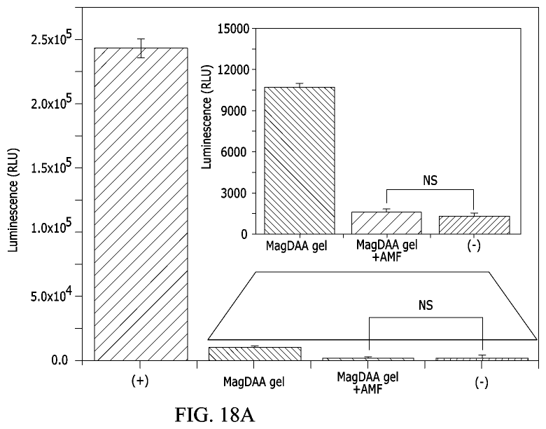

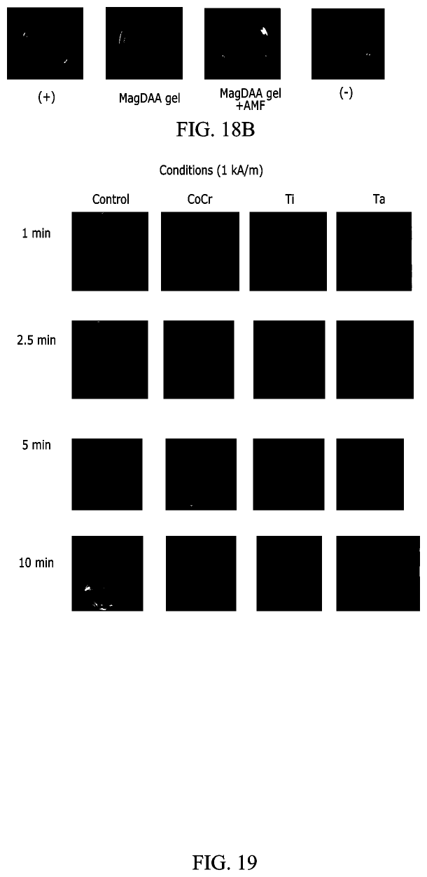

To further investigate whether the biofilms have been completely eradicated or there are remaining viable bacterial cells that could reattach to the surface and seed new biofilm formation, we have performed a microbial cell viability assay that provides a method to determine the number of viable bacterial cells based on the quantitation of the ATP present. This assay involves adding a commercial reagent (BacTiter-Glo Reagent, Promega) directly to the bacterial cells released into the test medium and measuring luminescence to evaluate if the cells that have been freed from the biofilms following D-AA or combined D-AA+AMF treatments are viable or not. Shown in FIG. 18A is the result of our microbial cell viability assay that demonstrates that the bacterial cells exposed to the combined D-AA and AMF-induced hyperthermia treatment are not viable, which would prevent them from reattaching to the surface to form new biofilms. On the other hand, to investigate whether there are adherent bacterial cells remaining that could regrow the biofilms, we performed a set of experiments to test the survival of remaining adherent bacterial cells using the colony-forming assay approach. Our results demonstrate that no new colonies can be formed from the biofilms subjected to the combined D-AA and AMF-induced hyperthermia treatments, while some colonies formed in the biofilms treated with only D-AA (FIG. 18B). These results demonstrate the potential viability of the two-step biofilm treatment approach, which utilizes a magnetically actuated gel nanocomposite system for thermal treatment that follows an initial disruption treatment with D-AAs, for complete biofilm eradication in the clinical setting.

### Example 2

The effects of magnetic hyperthermia conditions were studied using chicken breast cut into 35×20×10 mm (length×width×height) pieces and the different metal implant materials, cobalt chromium (CoCr), titanium (Ti), and tantalum (Ta) that are circular in shape with a diameter of 10 mm, where embedded in it. The chicken embedded with the implant materials were then exposed to magnetic hyperthermia treatments using a water cooled magnetic coil that generates alternating current (AC) field at different applied magnetic field amplitudes (H) (1, 2.5, and 5 kA/m) at a constant frequency (f) of 380 kHz. Different time exposures were also evaluated (1, 2.5, 5, and 10 min). Also, the temperature increase and change of temperature (ΔT) of cubic shaped iron oxide nanoparticles were measured using the same conditions.

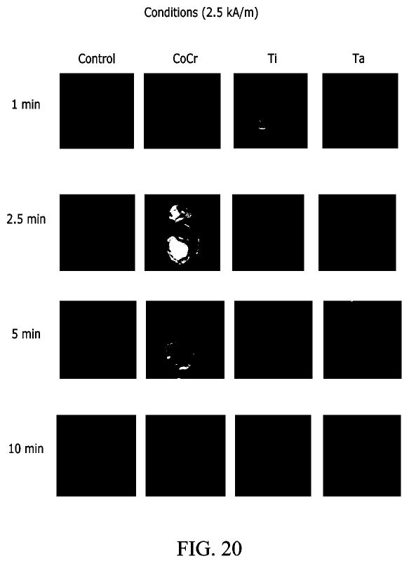

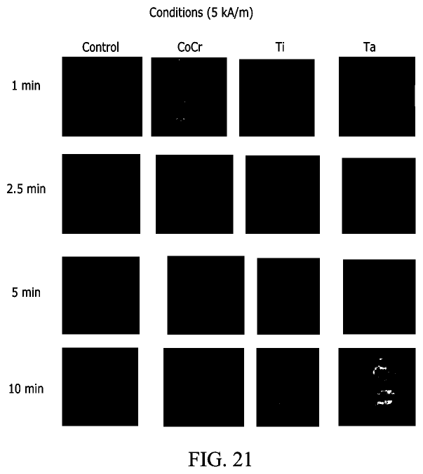

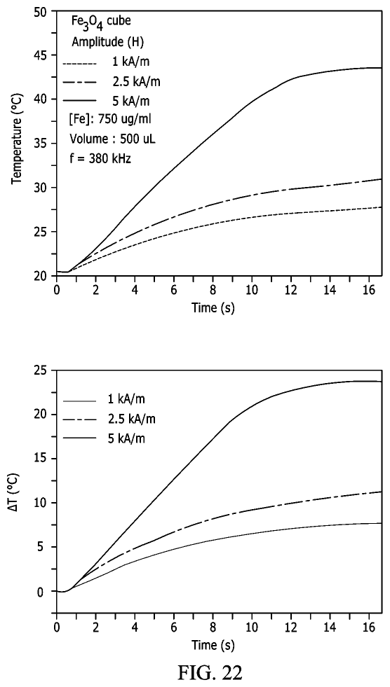

The results of effect of magnetic hyperthermia conditions on chicken meat are shown in FIGS. 19-22 and the quantitative measurements of burnt or heat affected area are summarized in Table 1.

TABLE 1
Thickness and diameter of raw chicken meat that
turned white/burned after magnetic hyperthermia exposure

| Amp- | | Heating | | Cobalt | Ti- | |
|---|---|---|---|---|---|---|
| litude | Time | effect | | chromium | tanium | Tantalum |
| (H) | (min) | (mm) | Control | (CoCr) | (Ti) | (Ta) |
| 1 kA/m | 1 min | Diameter | 0 | 10 | 10 | 0 |
| | | Thickness | 0 | 0.5 | 0.5 | 0 |
| 2.5 kA/m | 1 min | Diameter | 0 | 14 | 13 | 0 |
| | | Thickness | 0 | 1 | 1 | 0 |
| 5 kA/m | 1 min | Diameter | 0 | 18 | 16 | 3 |
| | | Thickness | 0 | 5.5 | 5 | 1 |
| 1 kA/m | 2.5 min | Diameter | 0 | 10 | 10 | 0 |
| | | Thickness | 0 | 1 | 0.75 | 0 |
| 2.5 kA/m | 2.5 min | Diameter | 0 | 15 | 14 | 0 |
| | | Thickness | 0 | 2 | 1.75 | 0 |
| 5 kA/m | 2.5 min | Diameter | 0 | 18 | 16 | 5 |
| | | Thickness | 0 | 7.5 | 6.8 | 1.5 |
| 1 kA/m | 5 min | Diameter | 0 | 10 | 10 | 0 |
| | | Thickness | 0 | 1.5 | 1 | 0 |
| 2.5 kA/m | 5 min | Diameter | 0 | 12 | 11 | 10 |
| | | Thickness | 0 | 5 | 3.4 | 0.5 |
| 5 kA/m | 5 min | Diameter | 0 | 20 | 20 | 12 |
| | | Thickness | 0 | 10 | 10 | 5 |
| 1 kA/m | 10 min | Diameter | 0 | 15 | 15 | 10 |
| | | Thickness | 0 | 5 | 5 | 2.5 |
| 2.5 kA/m | 10 min | Diameter | 0 | 15 | 15 | 10 |
| | | Thickness | 0 | 10 | 9 | 3 |
| 5 kA/m | 10 min | Diameter | 0 | 20 | 20 | 12 |
| | | Thickness | 0 | 15 | 15 | 5 |

According the results, the magnetic hyperthermia conditions have caused adverse effects on chicken meat with all the implant materials during 10 minutes of exposure time.

### Example 3

The effects of laser power on heating were studied in 0.9% saline solution and the different metal implant materials, CoCr, Ti, and Ta that are circular in shape with a diameter of 10 mm immersed in 0.9% saline solution separately. During the study, a 808 nm laser with laser power of 0.5, 0.8, and 2 W/cm² was used over 5 and 10 min of continuous exposure time. The actual temperature was monitored using a Neoptix temperature sensor. Further, the effect of laser on chicken breast was studied to compare that of magnetic hyperthermia conditions used in Phase I. Briefly, chicken breast cut into 25×20×5 mm (length×width×height) pieces and the three types of implants were embedded in it and then exposed to the laser with power of 0.8 W/cm² for 10 minutes continuously.

### Synthesis of Gold Nanorods (AuNRs) and Plasmon Band Fine Tuning to 810 nm Laser Excitation Source

### Synthesis of Au Seeds

For the synthesis of Au NRs, Au seeds (3-4 nm) were first synthesized by the chemical reduction of chloroauric acid (HAuCl₄) with a strong reducing agent sodium borohydride (NaBH₄) in the presence of a capping agent, cetyltrimethylammonium bromide (CTAB). The first solution was prepared by mixing a HAuCl4 (0.5 mM, 5 mL) solution with a CTAB solution (0.2 M, 5 mL) in a 20 mL scintillation vial. Simultaneously, a solution of NaBH₄ (0.01 M, 0.6 mL) was prepared. The NaBH₄ solution was injected into the Au (III)-CTAB solution under stirring (1200 rpm) for 2 min. Finally, the solution was aged at room temperature for 30 min before use (Ye, X. et al., Nano Lett. 2013, 13, 765-771).

### Synthesis of Au NRs

0.18 g of CTAB and 0.0269 g of sodium oleate (NaOL) were dissolved in 5 mL of warm water (50° C.) in a 20 mL scintillation vial. The solution was allowed to cool down to 30° C. before the addition of a silver nitrate solution (4 mM, 360 μL). The solution was left undisturbed for 15 min. Then a solution of HAuCl₄ (1 mM, 5 mL) was added under stirring (700 rpm) for 1.5 h. After that time, the pH of the solution was adjusted to 3-5 (depending of on the desired aspect ratio of the NRs), under constant stirring (400 rpm) for 15 min. Then a solution of ascorbic acid (0.064 M, 25 μL) was added under fast stirring (1200 rpm) for 30 s. Finally, the seeds solution (4 μL) was added at 1200 rpm for 3 is. The seeds solution served as nucleation sites for the anisotropic growth of Au NRs. The solution was left to react undisturbed for 12 h and the final product was centrifuged (7,000 rpm, 30 min). The supernatant was removed, and the particles were re-dispersed in water (Wu, W. et al., Chem. Mater. 2015, 27, 2888-2894).

TEM Sample Preparation

A diluted sample of Au NRs was prepared and 10 μL were deposited by gravity on a copper grid. The sample was left to dry at room temperature for 3 h before performing the analysis.

Study of Heating Profiles of AuNRs

The heating and cooling profiles of AuNRs in 0.9% saline solution were studied using the laser power of 0.8 W/cm². The results were compared with that on magnetic nanoparticles used in Example 2.

Study of AuNRs and D-Amino Acids (D-AA) Treatments of S. aureus Biofilm

Biofilm Formation and Dispersal Assays

S. aureus (ATCC 49525) were cultured overnight in kanamycin sulfate (200 ug/ml) containing luria broth (LBK) with agitation (200 rpm) at 37° C. Biofilm formation was evaluated under static conditions with or without titanium implant in polystyrene petri dish (fisherbrand). Bacterial cultures made overnight were further diluted in LBK broth for desired cell amount (2×10⁵ cfu/ml) at OD600 and each dish was filled with 3 mL of the diluted bacterial solution and incubated at 37° C. for 24 hr, with low agitation (100 rpm). Biofilm dispersal activities were evaluated by removing the culture media from the biofilms after 24 h and replacing it with saline solution (0.9% NaCl) containing D-amino acid mixture at 200 mM (pH 7.4), either with Au nanorods or without. Media for controls was replaced with saline solution and incubated as treatment. After exposure to the amino acid mixture, the plates were gently washed with phosphate buffered saline (PBS, 1×) thrice and colony counting and/or crystal violet stain was performed as needed.

Crystal Violet Stain

Once washed with PBS saline, remained biofilm was stained with 1 ml of crystal violet (0.01%) for 30 min. Liquid solution was removed and remaining stain was solubilized with 30% acetic acid. The solution was diluted 20 times and biofilm Biomass was estimated by measuring the crystal violet stained biofilm remnants, which absorbs at 595 nm.

Swab and Colony Counting

Followed by washing process with PBS, prior to stain, side of implant or bottom part of the petri dish was swabbed with sterile cotton swab and incubated in 5 ml LBK for 5 min. Cotton swab was removed and LBK solution was shaken for overnight (200 rpm, 37° C.). On the following day, 2.5 ul was taken out and diluted to 100 ul with LBK to be evenly plated on kanamycin-containing (200 ug/ml) agar gel plate. The plate was incubated overnight or longer to check any regrowth at 37° C.

Establishing Parameters for In Vitro Studies

The treatment parameters were systematically established by controlling each component of the study, which includes AuNRs formulation, optimizing AuNR dimensions and optical properties, ligand exchange process, heating profiles, and finally the complete eradication of S. aureus biofilm grown on Ti implant with the optimized conditions.

Surface Functionalization: Ligand Exchange

A solution of Au NRs (3.5 mM, 2 mL) was mixed with a solution of m-PEG-Thiol (1 M, 2 mL) for 1 h under constant stirring (1200 rpm). The final product was centrifuged, re-dispersed in saline solution and stored in the dark at 4° C. until use.

Atomic Absorption Spectroscopy (AAS): Determination of Au NRs Concentration

For total Au concentration, 10 μL of Au NRs samples were digested in 1 mL of HCl (12M) for 24 h and dilute in milli-Q water to 10 mL. The solutions were vortex mix and analyzed for Au using flame AAS using a standard calibration curve.

Surface Functionalization: Ligand Exchange

A thermoresponsive hydrogel based on glycol chitosan was prepared according to a previously reported procedure (Li, Z. et al., Carbohydr. Polym. 92 (2), 2267-2275). Briefly, glycol chitosan (0.25 g) was dissolved in a 50:50 mixture of water to methanol (50 mL), followed by the slow addition of acetic anhydride (0.83 mL). The resulting reaction mixture was then left to stir for 48 h. The product was then precipitated with acetone and centrifuged at 17000 rpm for 10 min at 4° C. The isolated glycol chitin was then redissolved in deionized water and incubated in 1 M NaOH solution for 12 h to remove undesired reaction byproducts. The sample was then dialyzed in a dialysis membrane (molecular weight cut off=2000 Da) for 3 days. The purified product was then reprecipitated with acetone and centrifuged at 4° C. to isolate the glycol chitin. Following lyophilization, the product was collected and stored for further use.

Treatment of S. aureus Biofilm Grown on Ti Implant

Briefly, S. aureus biofilm (2×10⁵ cfu/ml) was grown on Ti implant (circular disk with diameter of 10 mm) over 24 h. Then, it was incubated 37° C. for 2 h with 200 μL of 200 mM D-AA in 0.9% saline and AuNRs with different concentrations (500, 750, and 1000 ppm) loaded in 8% glycol chitin gel. To keep environment moist 0.9% saline solution added (300 μl) around the implant and 2 drops of saline (20 μL in total) were dropped onto the Au-nanogel composite at the beginning of incubation. Then, it was exposed to 20 minutes of 808 nm laser light (laser spot size is 1 cm² and laser power is 1 W/cm²) in 15 seconds laser pulses. The gap between the laser and biofilm was 4 cm. The results of treatments were compared with a positive and negative control using scanning electron microscopy and colony counting tests.

Results

Effect of Laser Conditions

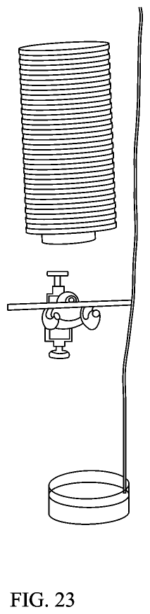

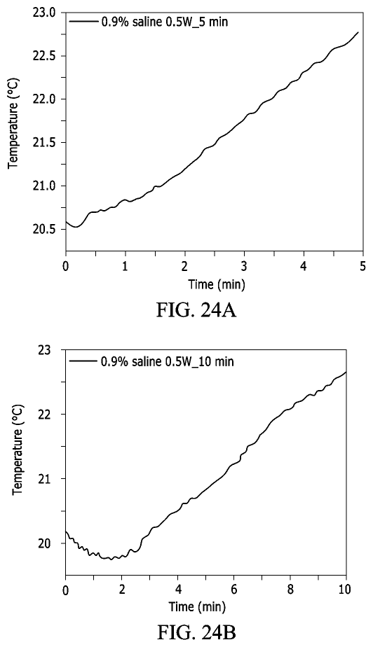

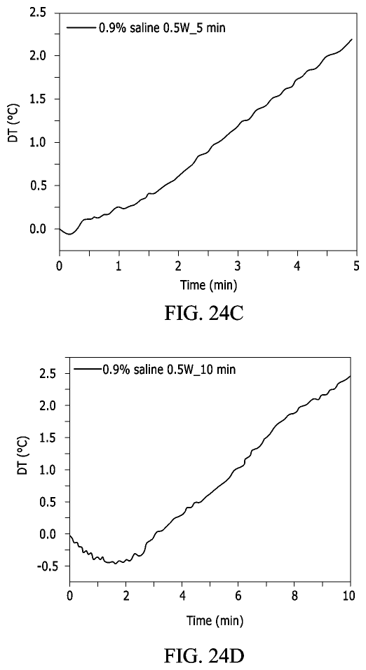

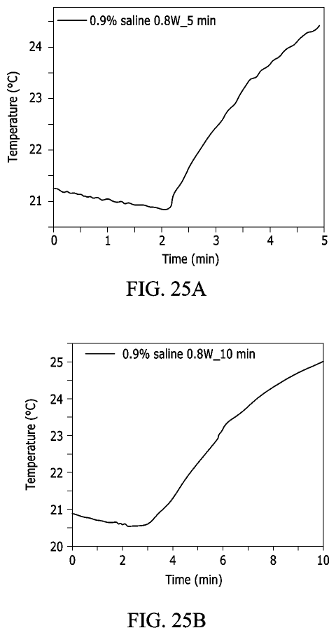

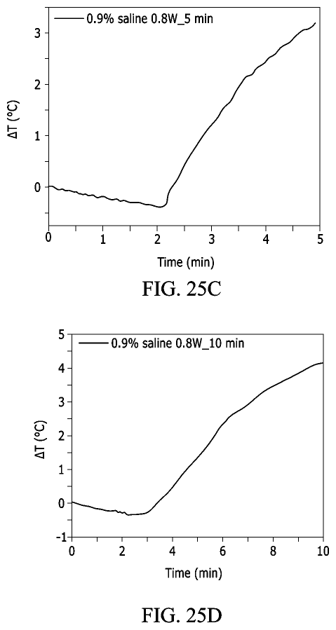

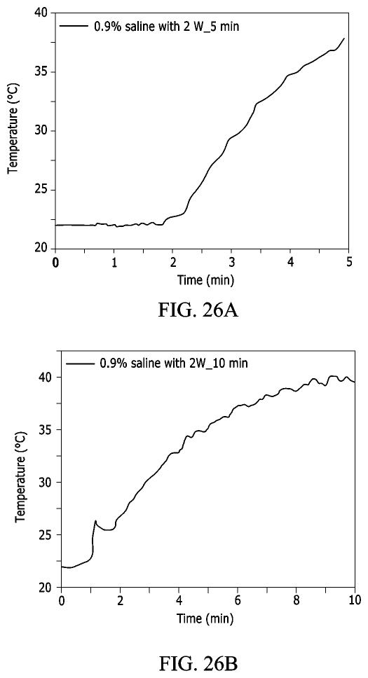

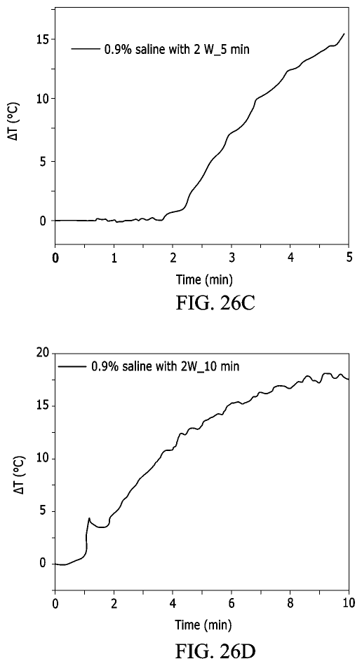

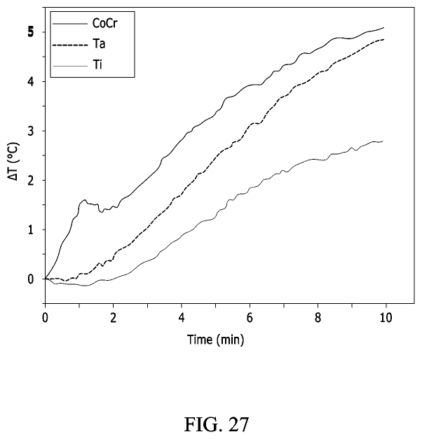

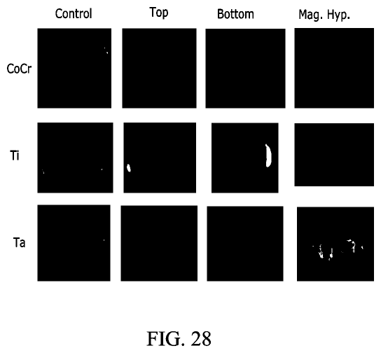

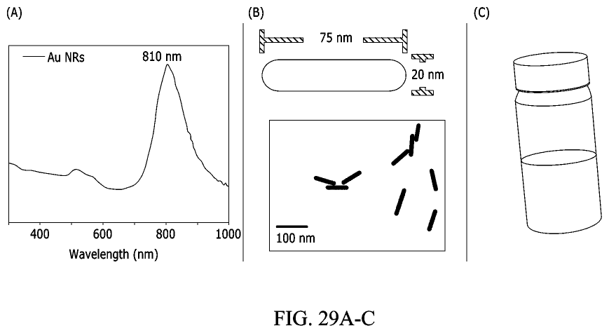

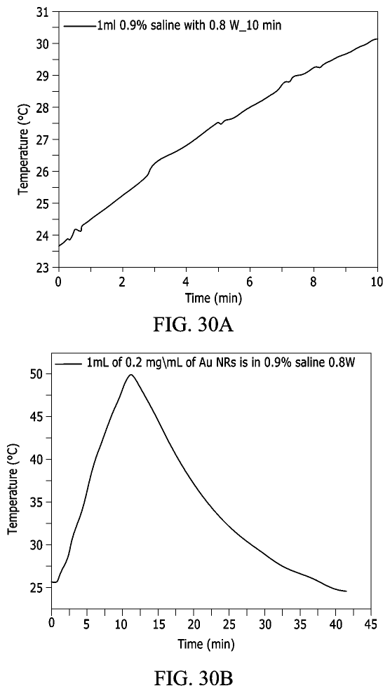

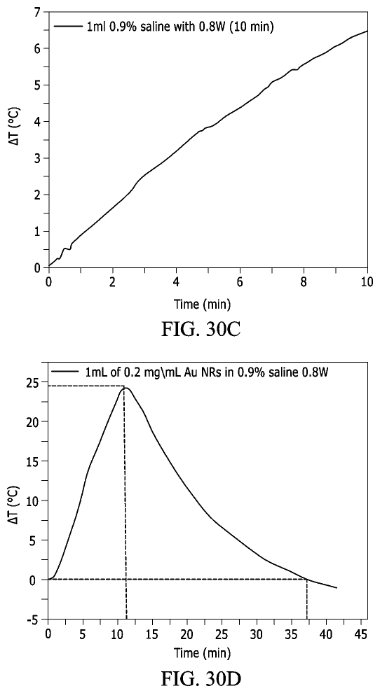

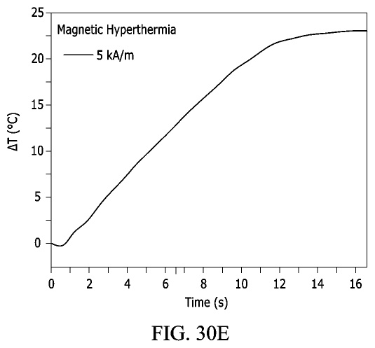

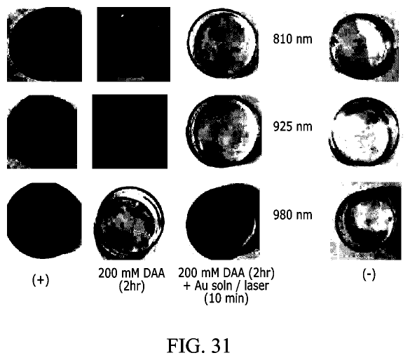

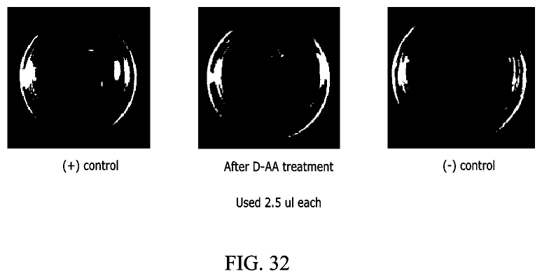

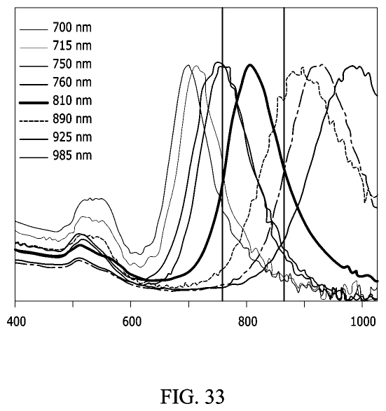

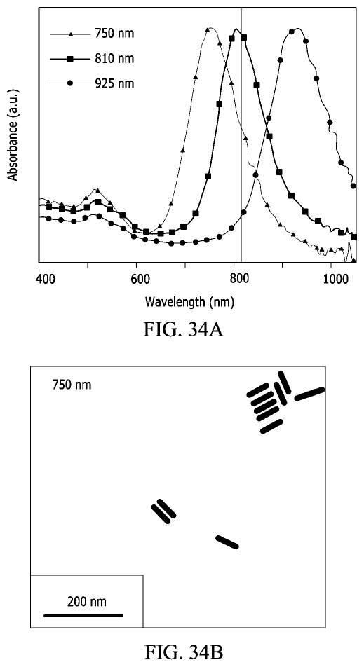

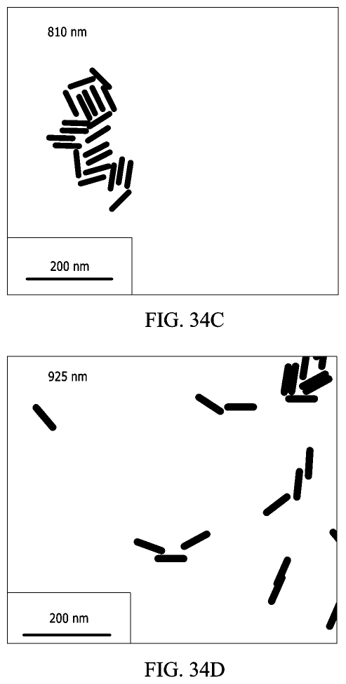

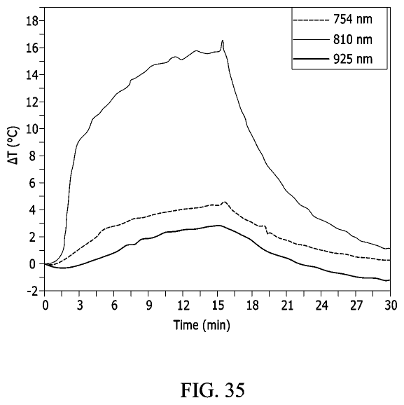

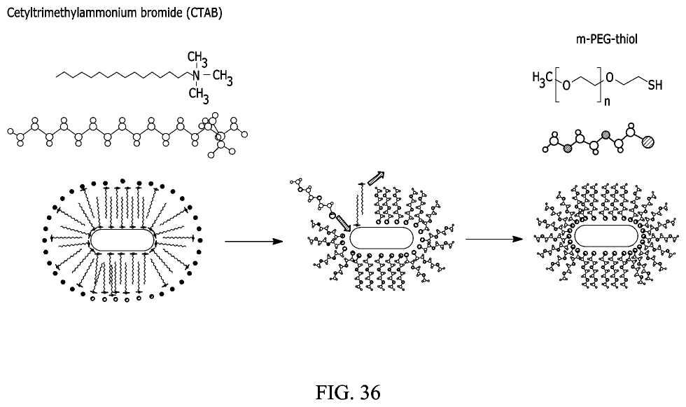

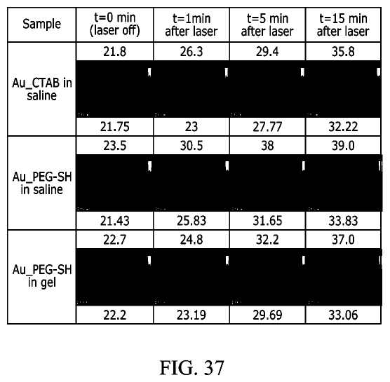

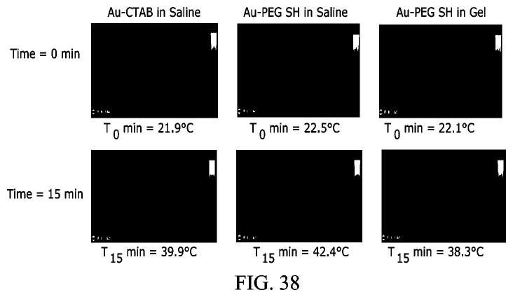

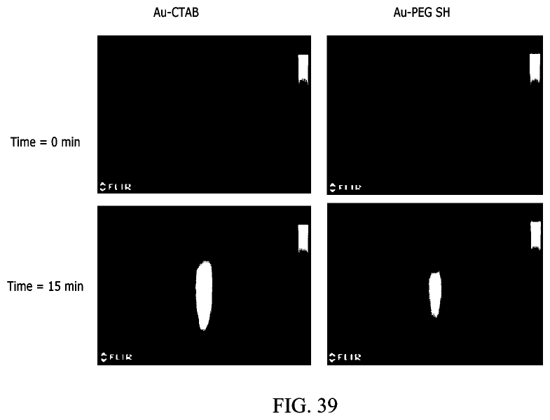

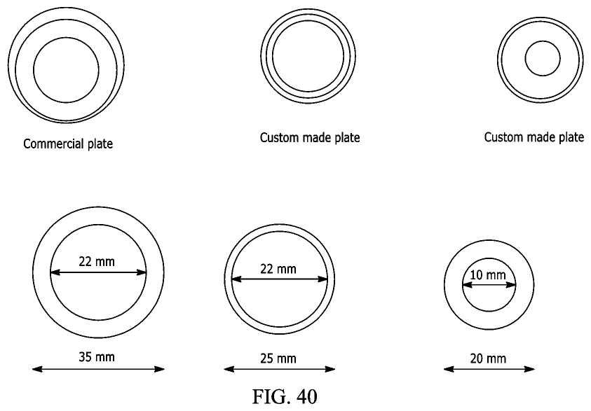

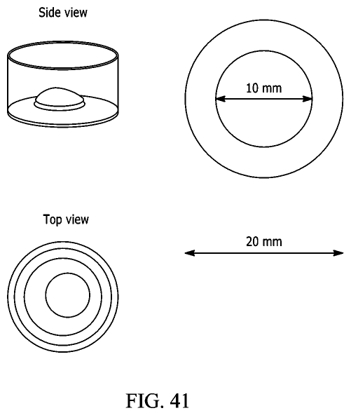

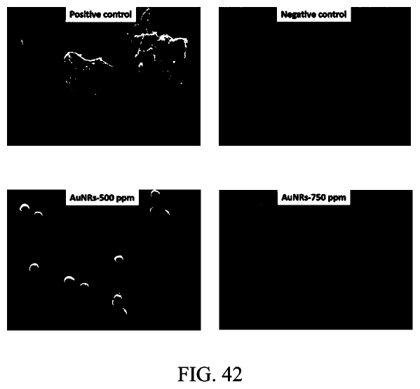

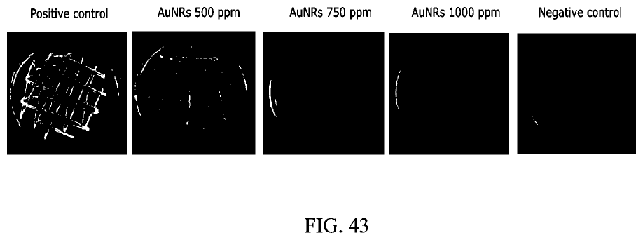

The effect of the 808 nm laser used in Phase II was systematically studied using the experimental setup shown in FIG. 23. The heating profiles of 0.9% saline solutions with different laser conditions are shown in FIGS. 24-26. Contrast to magnetic hyperthermia conditions used in Example 2, the implant materials show no interactions with the laser power we used over 10 minutes of continuous exposure time, which was further confirmed with chicken meat study (FIGS. 27-28). Additional study parameters and results are shown in FIGS. 29A-43. These results indicate that the laser conditions we adapted on photothermal approach is safe to use in in vivo conditions without any adverse effect on normal tissues.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A thermoresponsive nanocomposite for disrupting or preventing biofilm formation, the nanocomposite comprising:
at least one thermoresponsive polymer;
one or more D-amino acids; and
one or more energy-actuatable particles;
wherein the nanocomposite has a first viscosity at about room temperature and, when exposed to about physiological temperature, obtains a second viscosity that is greater than the first viscosity;
wherein application of energy to the nanocomposite from an energy source excites the one or more energy-actuatable particles to cause localized heat release from the nanocomposite.

2. The nanocomposite of claim 1, wherein the nanocomposite is a hydrogel.

3. The nanocomposite of claim 2, wherein the hydrogel is a glycol chitin-based hydrogel.

4. The nanocomposite of claim 1, wherein the one or more D-amino acids comprise a mixture of D-tyrosine, D-tryptophan and D-phenylalanine.

5. The nanocomposite of claim 1, further including one or more antibiotics.

6. The nanocomposite of claim 1, wherein the one or more energy-actuatable particles include magnetic nanoparticles, plasmonic nanoparticles, and combinations thereof.

7. The nanocomposite of claim 1, wherein the energy applied to the nanocomposite is light or a magnetic field.

8. A medical implant that is resistant to biofilm formation, wherein the medical implant is at least partially coated or impregnated with the nanocomposite of claim 1.

9. A method for disrupting or preventing biofilm formation on a surface, the method comprising applying energy from an external energy source to a surface that is at least partially coated or impregnated with the nanocomposite of claim 1, wherein the energy is applied for a time and in an amount sufficient to excite one or more energy-actuatable particles of the nanocomposite and cause localized heat release from the nanocomposite.

10. The method of claim 9, wherein the nanocomposite is a hydrogel.

11. The method of claim 10, wherein the hydrogel is a glycol chitin-based hydrogel.

12. The method of claim 9, wherein the one or more D-amino acids comprise a mixture of D-tyrosine, D-tryptophan and D-phenylalanine.

13. The method of claim 9, wherein the nanocomposite further include one or more antibiotics.

14. The method of claim 9, wherein the one or more energy-actuatable particles include magnetic nanoparticles, plasmonic nanoparticles, and combinations thereof.

15. The method of claim 9, wherein the energy applied to the nanocomposite is light or a magnetic field.

16. A method for disrupting or preventing biofilm formation on an in situ medical implant, the method comprising:
exposing a surface of the medical implant;
contacting, at about room temperature, the surface of the medical implant with a nanocomposite having a first viscosity, the nanocomposite comprising at least one polymer, one or more D-amino acids, and one or more energy-actuatable particles;
allowing a period of time to pass so that the nanocomposite obtains a second viscosity that is greater than the first viscosity;
applying, to the nanocomposite, energy from an energy source in an amount and for a time sufficient to excite one or more energy-actuatable particles of the nanocomposite and cause localized heat release from the nanocomposite; and
covering the surface of the implanted medical implant.

17. The method of claim 16, wherein the nanocomposite, when contacted with the surface at about physiological temperature provides sustained release of the one or more D-amino acids to disrupt or prevent biofilm formation.

18. The method of claim 16, wherein the localized heat release from the nanocomposite substantially eradicates remaining attached bacterial cells and planktonic bacterial cells released from the disrupted biofilm.
